(19) United States
(12) Patent Application Publication
(10) Pub. No.: US 2019/0211066 A1
(43) Pub. Date: Jul. 11, 2019

(54) VACCINES FOR PREVENTING MENINGOCOCCAL INFECTIONS

(71) Applicants: Mathieu COUREUIL, Paris (FR); Xavier NASSIF, Paris (FR); Stefano MARULLO, Paris (FR)

(72) Inventors: Mathieu COUREUIL, Paris (FR); Xavier NASSIF, Paris (FR); Stefano MARULLO, Paris (FR)

(73) Assignees: INSTITUT NATIONAL DE LA SANTE ET DE LA RECHERCHE M EDICALE (INSERM), Paris (FR); UNIVERSITE PARIS DESCARTES, Paris Cedex 06 (FR)

(21) Appl. No.: 16/360,085

(22) Filed: Mar. 21, 2019

Related U.S. Application Data
(63) Continuation of application No. 15/702,817, filed on Sep. 13, 2017, now abandoned.
Application No. 15/702,817 is a continuation of application No. 15/064,857, filed on Mar. 9, 2016, now abandoned.
Application No. 15/064,857 is a continuation of application No. 13/883,328, filed on Jul. 10, 2013, now abandoned.

(30) Foreign Application Priority Data
Nov. 5, 2010 (EP) 10306215.4

(51) Int. Cl. C07K 14/22; A61K 38/16; A61K 47/68; A61K 47/64; A61K 39/095; C07K 16/28; C07K 14/705

(52) U.S. Cl. CPC C07K 14/22; A61K 38/164; A61K 47/6811; A61K 47/64; A61K 38/00; A61K 47/6849; C07K 16/286; C07K 14/70571; A61K 39/095

(57) ABSTRACT

The present invention stems from the finding that the interaction between the β2 adrenoceptor (β2AP) and type IV pilus-associated proteins initiates a process leading to the opening of the blood-brain barrier The invention therefore pertains to a vaccine for preventing the spreading of meningococci into the meningcal space, wherein said vaccine allows the production of antibodies inhibiting the interaction between the type IV pilus-associated proteins and the β2AP.

The present invention stems from the finding that the interaction between the β2 adrenoceptor (β2AR) and type IV pilus associated proteins initiates a process leading to the opening of the blood-brain barrier. The invention therefore pertains to a vaccine for preventing the spreading of meningococci into the meningeal space, wherein said vaccine allows the production of antibodies inhibiting the interaction between the type IV pilus-associated proteins and the β2AR.

Meningococcus (N. meningitidis, Nm), a cause of epidemic meningitis and sepsis, is a commensal Gram-negative bacterium of the human nasopharynx. After bloodstream invasion, virulent encapsulated bacteria adhere to brain endothelial cells and proliferate onto the apical surface of host cells to form microcolonies at the site of initial bacterial attachment, then cross the Blood Brain Barrier (BBB) to colonize meninges (Nassif et al., 2002, Trends Microbial 10, 227-232).

Both bacterial adhesion and subsequent Nm-promoted signaling in host cells depend on the presence of bacterial filamentous structures, the type IV pili (tip) (Nassif et al., 1994, Proc Natl Acad Sci USA 91, 3769-3773; Virji et al., 1991, Mol Microbial 5, 1831-1841) and of cellular receptor(s), which still remain elusive.

In vitro experiments under controlled flow indicated that meningococcal adhesion could only occur at low shear stress levels, which are observed in capillaries upon transient reduction in flow. After initial attachment, bacteria acquire the capacity to resist higher blood velocities and to multiply, forming colonies. Cellular and molecular mechanisms of this tighter adhesion are initiated by tfp-dependent activation of host cell signaling cascades. In vitro, encapsulated meningococci elicit the organization of specific “honeycomb”-shaped molecular complexes underneath bacterial colonies, referred to as “cortical plaques”. Cortical plaques result from the recruitment of molecular linkers, such as ezrin and moesin, adhesion molecules and membrane receptors, and from localized polymerization of cortical actin, leading to the formation of microvilli-like plasma membrane protrusions among bacteria, which are crucial for Nm colonies to resist shear stress and colonize blood vessels. Changes of the host cell cytoskeleton induced by Nm are associated with the turning on of multiple signaling events including the activation of the Src kinase, the activation of the GTPases Rho, Cdc42, and Rac, the PI3-kinase-mediated recruitment of cortactin and the activation of mitogen-activated kineses.

Whether Nm ultimately pass through the barrier of brain endothelial cells by transcytosis (Massif et al., 2002, Trends Microbiol 10, 227-232) or through cell junctions has been debated for a long time. Recently, however, Nm were shown to recruit endothelial cell adhesion molecules, such as VE-cadherin, into cortical plaques. This recruitment leads to the formation of ectopic intercellular junctional domains at the site of bacteria-host cell interaction, depletion of junctional proteins at the cell-cell interface, opening of intercellular junctions and subsequent crossing of the BBB (Coureuil et al., 2009, Science 325, 83-87).

Pleiotropic signaling events elicited by Nm in endothelial cells, which allow bacteria to resist to blood flow and cross the BBB, might either proceed from independent parallel signaling pathways, or, more likely, depend on a multimolecular signaling complex scaffolded downstream of an single host cell receptor. Among candidate proteins capable of scaffolding multiple cellular signals, β-arrestins (referred here to βarr1 and βarr2), two ubiquitously expressed molecules initially described as regulators of G protein coupled receptors (GPCRs), participate in many of the signaling pathways that are elicited by meningococci. For example, βarrs operate as adaptor proteins to recruit and activate Src under agonist-occupied receptors. In addition, βarrs act as scaffolds for GPCR-mediated activation of the MAPKs ERK1/2 into cytoplasmic compartments, and drive localized activation of cortical actin polymerization. βarrs were also shown to contribute to the activation of several Ras-family GTPases. Finally. the junctional depletion of adhesion molecules with subsequent enhanced vascular permeability, promoted by activation of the vascular endothelial growth factor (VEGF), involves βarr-dependent endocytosis of phospho-VE-cadherin into clathrin-coated vesicles.

However, the cellular receptor which allows internalization of N. meningitis by opening the intercellular junctions or initiating transcytosis still remains unknown. The tip proteins involved in the interaction with that cellular receptor are also unknown.

Crossing of the blood-brain barrier (BBB) is a burning problem in the field of medicine. Indeed, the major problem encountered when wanting to treat CNS disorders is how to deliver a therapeutic agent into the brain.

It has been shown that a direct, topical application of a β-adrenoceptor agonist to the brain paremchyma increased the permeability of the BBB (Chi et al. 1998, Neurol Res. 20:259-64). However, Chi et al. do not teach whether increased BBB permeability is a direct or an indirect effect of β-adrenoceptor activation, and fail to elucidate which β-adrenoceptors are involved in the observed increase of BBB permeability. In addition, Chi et al. neither teach nor suggest any mean allowing a systemic administration of a therapeutic agent.

The main clinical feature of meningococcal infection is meningitis, Paradoxically, Neisseria meningitidis is a frequent asymptomatic colonizer of the human nasopharynx, and only a very small proportion of infections proceed to a sustained bacteraemia. In the majority of bacteremic patients the presence of N. meningitdis in the bloodstream will remain asymptomatic. However during the blood phase, the bacteria will interact with the brain endothelial cells and invade the meninges to be responsible for meningitis which is the first clinical complication of meningococcal dissemination in 70% of patients. In 30% of bacteremic patients the presence of meningococci in the bloodstream will lead to clinical symptoms of septicemia with in some cases a purpura fulminans. In the latter case the bacteria cross the blood brain barrier; however, the meningitis symptoms are not at the forefront of the clinical presentation.

The reasons why meningococcal disseminate from the nasopharynx into the bloodstream in some individuals and not in others remain unclear, but human genetic polymorphism is likely to be important in determining the outcome of nasopharyngeal colonization. In addition, all meningococci do not have the same pathogenic potential. Indeed, analysis of results from multilocus sequence typing (MLST) have demonstrated the existence of distinct phylogenetic groups (clonal complexes), some of which are more likely to be isolated from patients than others. These are the so-called “hyper-virulent” or “hyperinvasive” lineages. Recently, the presence of a prophage has been shown to be responsible for a large proportion of invasiveness of strains belonging to hyperinvasive lineages. This element inserted into the chromosome can be induced to produce a filamentous phage.

Few bacterial pathogens have the ability to cross the blood brain barrier and to invade the meninges. After the neonatal period those pathogens are, beside Neisseria meningitdis, mostly Streptocccus pneumoniae, Haemophilus influenzae. During the neonatal period Streptococcus agalactiae and Escherichia coil are routinely isolated. Finally, at all ages but rarely and in some circumstances Mycobacterium tuberculosis and Listeria monocytogenes are responsible for meningitis. However among those pathogens the one that achieves this goal the most constantly and the most efficiently is Neisseria meningitidis, thus pointing out the efficiency with which this pathogen can cross the blood brain barrier. A study using postmortem examination has shown that meningeal invasion is a consequence of the direct interaction of N. meningitidis with the brain endothelial cells (Pron et al, Journal of Infectious Diseases, 1997, 176(5):1285-92). In vitro experiments using a brain endothelial cell line has shown that the type IV pili of N. meningitidis are responsible for the formation of microcolonies on the apical surface of endothelial cell capillaries. The formation of these microcolonies leads to a signalling to the endothelial cells and the recruitment of junctional molecules at the site of the bacterial cell interaction, thus depleting the intercellular junctions and opening the paracellular route. N. meningitidis can then cross this monolayer between two adjacent cells.

A vaccine aiming at protecting against cerebrospinal meningitis will be best if it prevents bacterial dissemination in the bloodstream by inducing bactericidal antibodies and inhibits the ability of the bacteria to cross the blood brain barrier. Most of the current strategies aiming at engineering a vaccine against rneningococcal infection concentrate solely on the production of bactericidal antibodies. Considering that the dissemination of the bacteria into the blood remains asymptomatic until it crosses the blood brain barrier, preventing the ability of the bacteria to signal to brain endothelial cells will prevent meningeal dissemination and bacterial meningitis. The inclusion in a meningococcal vaccine of epitopes inducing antibodies preventing meningococcal crossing of the blood brain barrier will therefore be beneficial for the prevention of cerebrospinal meningitis.

Thus there is a need for vaccines preventing the meningococci to cross the blood brain barrier and consequently preventing cerebrospinal meningitis.

The inventors have surprinsingly found that the signalling leading to the opening of the blood brain barrier is a consequence of the interaction of two pilus components, the major pilin PilE and the minor pilin PilV, with the N-terminal portion of the beta2 adrenergic receptor. This interaction leads to the activation of the beta arrestin and the subsequent signalling into the host cells followed by the opening of the intercellular junction proteins.

Thus the inventors have found that a vaccine inducing antibodies inhibiting the interaction between the components of the type IV pill carrying the recognition domain to the beta2 adrenergic receptor responsible for meningococcal signalling, i.e. these components are the major pilin PilE and the minor pilin PilV, is particularly useful for preventing cerebrospinal meningitis. More particularly, they have discovered that purified PilE and PilV or portion of these peptides, used either in association, or fused in the same polypeptide chain to increase their immunogenicity, are the best candidate to induce the production of protective antibodies, as long as the fragment of each molecule encompass the domain of interaction with the beta2 adrenergic receptor.

The mature protein of PilE is approximately 145-160 residues long after processing by a prepilin peptidase. It has a conserved hydrophobic N-terminal 25 residue and a carboxy-terminal disulphide bond. The conserved hydrophobic N-terminal moiety that is embedded in the core of the pilus is involved in the cohesion of the subunits as a fibre. The polymorphic C-terminus of pilin is partially exposed at the surface of the assembled pilus. The pilin also harbours two cysteine residues that build a disulfide bridge, involved in the formation of the surface accessible, variable and immune dominant part of the pilus. Pilins are therefore packed through internal hydrophobic interactions between conserved N-terminal α helices, leaving hypervariable C-terminal globular regions exposed. The major pilin is encoded by the PilE gene. The fragment contained in the vaccine will therefore be either a complete pilin or a fragment of the molecule expressing the epitope binding to the N-terminal portion of the beta2 adrenergic receptor. In addition considering the great variability of pilin, different variants may be included in the vaccine. However, it is likely that the domain binding to the beta2 adrenergic receptor has a conserved structure between strains.

The minor pilin PilV is structurally very similar to the major pilin but these genes are present in every strain tested and are remarkably conserved between different serogroups and clonal complexes. As for the major pilin PilE, a fragment deleted of the N-terminal hydrophobic portion or even a smaller fragment can be used as long as antibodies against this fragment are capable of inducing antibodies inhibiting the endothelial cell signaling.

### DESCRIPTION OF THE INVENTION

The inventors have surprisingly found that Nm colonies at the cell surface of a human brain endothelial cell line promote the translocation of βarrs to the inner surface of plasma membrane, facing bacteria. βarrs translocated under the colonies serve as a scaffolding platform for signaling events elicited by Nm. Among the GPCRs expressed in the cell line, only the β2-adrenoceptor (β2AR) plays a permissive role in the formation of cortical plaques under colonies and in bacterial crossing of cell monolayers. These observations reveal the requisition of a β2-adrenergic/β-arrestin signaling pathway by Nm to promote stable adhesion onto human brain endothelial cells and subsequent crossing of the BBB.

More specifically, it has been shown that interaction of β2AR, in particular of the N-terminus of β2AR, with type IV pilus-associated proteins such as PilE or PilV, initiates the process allowing N. meningitidis to open and to traverse the BBB.

The inventors have surprisingly found that a vaccine inducing antibodies inhibiting the interaction between the components of the type IV pili carrying the recognition domain to the beta2 adrenergic receptor responsible for meningococcal signalling, i.e. major pilin PilE and minor pilin PilV, is particularly useful for preventing cerebrospinal meningitis.

### Definitions

By “PilE protein” and “PilV proteins”, it is meant PilE and PilV proteins derived from any meningococcal serogroup, e.g. from a meningococcus of serogroup A, B, C, V or W135. Preferably, said PilE and PilV proteins comprise or consist of:
a) the sequence of SEQ ID NO: 1 (N. meningitidis PilE);
b) the sequence of SEQ ID NO: 2 (N. meningitidis PilV); or
c) a sequence that is at least 80, 85, 90, 95 or 99% identical to the sequence of (a) or (b).

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95% or 99% identical” to a reference sequence may comprise mutations such as deletions, insertions and/or substitutions compared to the reference sequence. In case of substitutions, the protein consisting of an amino acid sequence at least 80%, 85%, 90%, 95% or 99% identical to a reference sequence may correspond to a homologous sequence derived from another species than the reference sequence. In another preferred embodiment, the substitution preferably corresponds to a conservative substitution as indicated in the table below.

| Conservative substitutions | Type of Amino Acid |
|---|---|
| Ala, Val, Leu, Ile, Met, Pro, | Amino acids with aliphatic hydrophobic |
| Phe, Trp | side chains |
| Ser, Tyr, Asn, Gln, Cys | Amino acids with uncharged but polar |
| | side chains |
| Asp, Glu | Amino acids with acidic side chains |
| Lys, Arg, His | Amino acids with basic side chains |
| Gly | Neutral side chain |

Mammalian β2 adrenoceptors (abbreviated β2ARs) are well known to the skilled in the art. For example, the human β2 adrenoceptor encompasses the protein having the sequence of SEQ ID NO: 3 as well as allelic variants and splice variants thereof. In the frame of the present invention, the mammalian β2AR may for example correspond to a human, a rat, a mouse, a guinea pig, a Chinese hamster, a rabbit or a monkey β2AR.

By “protective antibodies”, it is meant antibodies inhibiting the interaction between PilE and/or PilV and β2AR. More particularly, said antibodies inhibit the interaction between the N-terminus of β2AR and, PilE or PilV. Thus, said protective antibodies may for example recognize a linear epitope either comprised within the recognition domain to the β2AR of PilE or PilV, or comprising at least 1, 2, 3, 4, 5, 6, 7, 8, 9 or 10 consecutive amino acids located within the recognition domain to the β2AR of PilE or PilV. Alternatively, the antibody may bind to a conformational epitope comprising at least 1, 2, 3, 4, 5, 6, 7, 8, 9 or 10 amino acids located within the recognition domain to the β2AR of PilE or PilV.

By “Immunogenic fragment” is meant a contiguous portion of PilE or PilV which has the same or substantially the same immunogenic activity as PilE or PilV, respectively. That is to say, the fragment is capable of raising an immune response with antibodies that recognizes PilE or PilV. Preferably, the fragment is capable of raising an immune response with antibodies that prevent meningococcal infection. More preferably, said fragment is capable of raising an immune response with antibodies that prevent meningococci to cross of the BBB or to spread into the meningeal space. Still more preferably, said fragment is capable of raising an immune response with antibodies that inhibit the interaction between PilE and/or PilV and β2AR.

In a particular preferred embodiment, said immunogenic fragment does not carry the hydrophobic domain of PilE or PilV. More particularly, said immunogenic fragment of PilE does not carry the hydrophobic domain of sequence FTLIELMIVIAIVGILAAVALPAYQDYTARAQVSEAILLAEGQKSAVTEYYL (SEQ ID NO: 16). More particularly, said immunogenic fragment of PilV does not carry the hydrophobic domain of sequence FTLLELMIAVAILGILTLITYPSYKTYIRRVRLSEVRTTLLHNAQTMERYYRQ (SEQ ID NO: 17).

In another particular preferred embodiment, said immunogenic fragment or PilE comprises or consists of:
a) the sequence SAVTEYYLNHGEWPGDNSSAGVATSADIKGKYVQSVTVANGVITAQMASSNVNNEIKSK KLSLAKRQNGSVKWFCGQPVTRTTATATDVAAANGKTDDKINTKHLPSTCRDDSSAS (SEQ ID NO :18), or
b) a fragment of at least 10, 15, 20, 25, 30, 35, 40, 45, 50, 55, 60, 65, 70, 75, 80, 85, 90, 95, 100, 105, 110, 115 consecutives amino acids of the sequence SEQ ID NO :18, or
c) a sequence that is at least 80, 85, 90, 95 or 99% identical to the sequence of (a) or (b).

In another particular preferred embodiment, said immunogenic fragment or PilV comprises or consists of:
a) the sequence TMERYYRQKGTFKTYDKNKLKQNKYFNVTLSKVSPDHFTLQADPNPTTNDGETCVVTLD GGTIAASGTNQSCPGFD (SEQ ID NO:19), or
b) a fragment of at least 10, 15, 20, 25, 30, 35, 40, 45, 50, 55, 60, 65, 70, 75 consecutives amino acids of the sequence SEQ ID NO:1 9, or
c) a sequence that is at least 80, 85, 90, 95 or 99% identical to the sequence of (a) or (b).

In still another particular preferred embodiment, said immunogenic fragment carries the recognition domain to the β2AR responsible for meningococcal signaling.

More particularly, said immunogenic fragment of PilE comprises or consists of:
a) the sequence CGQPVTRTTATATDVAAANGKTDDKINTKHLPSTC (SEQ ID NO: 20), or
b) a fragment of at least 10, 15, 20, 25, 30 consecutives amino acids of the sequence SEQ ID NO:20, or
c) a sequence that is at least 80, 85, 90, 95 or 99% identical to the sequence of (a) or (b).

More particularly, said immunogenic fragment of PilV comprises or consists of:
a) the sequence GETCVVTLNDGGTIAASGTNQSCPGFD (SEQ ID NO:21), or
b) a fragment of at least 10, 15, 20, 25 consecutives amino acids of the sequence SEQ ID NO :21, or
c) a sequence that is at least 80, 85, 90, 95 or 99% identical to the sequence of (a) or (b).

By “meningococcal vaccine antigen” is meant a meningococcal antigen that is capable of raising an immune response against N. meningitidis, and that is suitable for use as a vaccine. As used herein, this term does not include PilE, PilV and immunogenic fragments thereof.

Such meningococcal vaccine antigens are well-known to the skilled in the art. Indeed, several vaccines against meningococci are commercialized and/or under development.

For example, the meningococcal vaccine antigen may correspond to one of the polysaccharide or glycoconjugate antigens comprised within the MCV-4 (commercialized under the name of Menactra® or Menveo®), MPSV-4 (Menomune®), Mencevax® or NmVac4-A/C/Y/W-135 vaccines.

The meningococcal vaccine antigen may also correspond to the capsule of group B meningococci (MenB) and/or to the outer membrane vesicle (OMV).

Preferably, the meningococcal vaccine antigen corresponds to a polypeptide. Several such polypeptidic antigens are currently investigated for their vaccinal potency. Thus the meningococcal vaccine antigen may for example be the Meningococcal Factor H binding protein (fHbp, previously referred to as GNA1870 or Lp2086), the major immunodominant surface porin (PorA), Neisserial Heparin Binding Antigen (NHBA, previously referred to as GNA2132), NadA, or immunogenic fragments thereof.

By “fHBP”, it is meant a fHBP polypeptide of subfamilyB/Variant1 or of subfamilyA/Variant 2. More particularly, said fHBP polypeptide may have the sequence SEQ ID NO: 22 or SEQ ID NO: 23 or a sequence at least 80, 85, 90, 95 or 99% identical to the sequence SEQ ID NO: 22 or SEQ ID NO: 23, or an immunogenic fragment thereof.

By “PorA”, it is meant a polypeptide that may have the sequence SEQ ID NO: 24 or a sequence at least 80, 85, 90, 95 or 99% identical to the sequence SEQ ID NO: 24, or an Immunogenic fragment thereof.

By “NHBA”, it is meant a polypeptide that may have the sequence SEQ ID NO: 25 or a sequence at least 80, 85, 90, 95 or 99% identical to the sequence SEQ ID NO: 25, or an immunogenic fragment thereof.

By “NadA”, it is meant a polypeptide that may have the sequence SEQ ID NO: 26 or a sequence at least 80, 85, 90, 95 or 99% identical to the sequence SEQ ID NO: 26, or an immunogenic fragment thereof.

“Immunogenic composition” as used in the context of the present invention relates to a composition comprising at least one protein or fragment thereof (hereafter referred to as “antigen”) which provokes or immunomodulates (i.e. immunosuppress or immunostimulate) an immune response when administered to an individual or which induces the production of antibodies when administered to an individual.

A “vaccine composition” as used herein refers to a composition, such as the immunogenic composition described herein which is administered to immunomodulate an immune response, that will protect or treat an individual from illness, in particular due to that agent. The vaccine of the present invention is intended for use both as a therapeutic (treatment) vaccine, i.e. for administration to the individual after development of the disease with the intention to reduce or arrest disease progression and as a preventive (prophylactic) vaccine, for administration to the individual prior to the development of the disease, with the intent to prevent initial (and/or recurrent) infection.

By “subject ” or “individual” it is meant a human, more preferably a men, a woman or a child.

### Vaccines According to the Invention

It has been shown that meningococcal PilE and PilV proteins are essential for N. meningococcus to cross the BBB and therefore to spread into the meningeal space. As a consequence, PilE, PilV and immunogenic fragments thereof can be used as a vaccine for preventing meningococcal infections. Indeed, administration of PilE, PilV and immunogenic fragments thereof will induce the production of antibodies inhibiting the interaction of PilE and/or PilV with β2AR, thereby preventing meningococci to cross of the BBB and to spread into the meningeal space.

Therefore, an aspect of the invention provides a meningococcal PilE or PilV protein, or an immunogenic fragment of at least 10, 20, 30, 40, 50, 60, 70, 80, 90, 100, 110, 120, 130, 140, 150 or 160 consecutive amino acids thereof, for use as a vaccine for preventing the spreading of meningococci into the meningeal space.

More preferably, said vaccine induces an immune response with antibodies preventing meningococcal infection, or preventing meningococci to cross of the BBB. Still more preferably, said vaccine induces the production of protective antibodies, i.e. antibodies inhibiting the interaction between PilE and/or PilV and β2AR.

In the frame of this aspect of the invention, the fragments of PilE or PilV are immunogenic fragments.

Methods for determining whether a fragment is immunogenic are well-known to the skilled in the art. For example, the capacity to raise an immune response against PilE or PilV may be tested using the assay described by Danve et al. (Vaccine. 1993;11:1214-20). Alternatively, the fragment to be tested may be used to raise specific antisera in a non-human animal such as a mouse. It may then be tested whether these antibodies are capable of inhibiting the interaction between meningococci and β2AR in vitro, e.g. using one of the protocols disclosed in the Examples.

As immediately apparent to the skilled in the art, in the frame of the aspect of the invention relating to vaccines, the fragments of PilE or PilV do not need to be capable of activating a mammalian β2 adrenoceptor. It is sufficient for them to be immunogenic. Preferably, the fragments of PilE or PilV need to be capable of raising an immune response with antibodies that prevent meningococcal infection, or that prevent meningococci to cross of the BBB or to spread into the meningeal space. More preferably, the fragments of PilE or PilV need to be capable of inducing the production of protective antibodies, i.e. antibodies inhibiting the interaction between PilE and/or PilV and β2AR.

The vaccine according to the invention is particularly useful for administration to individuals that are suspected of suffering from a meningococcal infection, but in whom the meningococci have not yet invaded the meningeal space. Such individuals include for example:
individuals suspected of presenting symptoms of a meningococcal infection;
individuals in which meningococci have been detected in the bloodstream but not in the cerebrospinal fluid;
individuals (preferably without symptoms of a meningococcal infection) who have traveled or who reside in countries with endemic or epidemic meningococcal disease (including Europe, the USA, China and Japan); and
individuals (preferably without symptoms of a meningococcal infection) who have been in contact with individuals with invasive meningococcal disease (e.g. relatives, schoolmates, medical staff, etc.).

In addition, PilV and PilE existing being present on all meningococcal groups and serotypes, the vaccine according to the invention is expected to prevent spreading of all meningococci, in particular of meningococci of group B.

PilE, PilV and immunogenic fragments thereof can be used in combination with a second meningococcal vaccine antigen.

Therefore, the invention also pertains to a combination of at least two of:
a meningococcal PilE protein, or an immunogenic fragment of at least 10 consecutive amino acids thereof,
a meningococcal PilV protein, or an immunogenic fragment of at least 10 consecutive amino acids thereof; and
a meningococcal vaccine antigen,
   wherein said meningococcal vaccine antigen is not PilE or PilV or an immunogenic fragment thereof.

For example, said combination comprises or consists of the immunogenic fragment of PilE of sequence SEQ ID NO: 18 and the fHBP polypeptide of sequence SEQ ID NO: 23, or of the immunogenic fragment of PilE of sequence SEQ ID NO: 20 and the fHBP polypeptide of sequence SEQ ID NO: 23.

For example, said combination comprises or consists of the immunogenic fragment of PilV of sequence SEQ ID NO: 19 and the fHBP polypeptide of sequence SEQ ID NO: 23, or of the immunogenic fragment of PilV of sequence SEQ ID NO: 21 and the fHBP polypeptide of sequence SEQ ID NO: 23.

Moreover, the invention pertains to a combination of at least two of:
a meningococcal PilE protein, or an immunogenic fragment of at least 10 consecutive amino acids thereof;
a meningococcal PilV protein, or an immunogenic fragment of at least 10 consecutive amino acids thereof; and
one or more meningococcal vaccine antigen(s);
   for simultaneous or sequential use as a vaccine for preventing a meningococcal infection, in particular for preventing the spreading of meningococci into the meningeal space.

More particularly, preventing the spreading of meningococci into the meningeal space may be performed by inhibiting the interaction between PilE and/or PilV and β2AR. Consequently, the use of said combination may induce the production of protective antibodies.

The combinations according to the invention may for example comprise or consist of:
a meningococcal PilE protein, or an immunogenic fragment of at least 10 consecutive amino acids thereof, and one or more meningococcal vaccine antigen, or
a meningococcal PilV protein, or an immunogenic fragment of at least 10 consecutive amino acids thereof, and one or more meningococcal vaccine antigen, or
a meningococcal PilE protein, or an immunogenic fragment of at least 10 consecutive amino acids thereof, and a meningococcal PilV protein, or an immunogenic fragment of at least 10 consecutive amino acids thereof, and one or more meningococcal vaccine antigen.

For example, said combination comprises or consists of the immunogenic fragment of PilE of sequence SEQ ID NO: 18 and the fHBP polypeptide of sequence SEQ ID NO: 23, or of the immunogenic fragment of PilE of sequence SEQ ID NO: 20 and the fHBP polypeptide of sequence SEQ ID NO: 23.

For example, said combination comprises or consists of the immunogenic fragment of PilV of sequence SEQ ID NO: 19 and the fHBP polypeptide of sequence SEQ ID NO: 23, or of the immunogenic fragment of PilV of sequence SEQ ID NO: 21 and the fHBP polypeptide of sequence SEQ ID NO: 23.

The said meningococcal vaccine antigens generally correspond to antigens having bactericidal activity, i.e. they which aim at generating an immune response with antibodies aiming at killing the meningococci. The combination according to the invention is expected to be a highly efficient vaccine since it would have two different actions: a bactericidal activity on the one hand (thanks to the meningococcal vaccine antigen), and prevention of the spreading of meningococci into the meningeal space on the other hand (thank to PilE, PilV or the immunogenic fragment thereof).

A particularly preferred aspect of the invention pertains to fusion proteins comprising at least two of:
a meningococcal PilE protein, or an immunogenic fragment of at least 10 consecutive amino acids thereof;
a meningococcal PilV protein, or an immunogenic fragment of at least 10 consecutive amino acids thereof; and
one or more meningococcal vaccine antigen(s).

Such fusion proteins are believed to be particularly suitable for use as a vaccine for preventing a meningococcal infection. Indeed, since they comprise at least two antigens that are fused together, they are highly immunogenic and thus highly efficient vaccines. In addition, they have the same advantages as the above combinations. More particularly, said fusion proteins have the ability to raise an immune response with antibodies that prevent meningococcal infection, or that prevent meningococci to cross of the BBB or to spread into the meningeal space. Still more particularly, said fusion proteins induce the production of protective antibodies inhibiting the interaction between PilE and/or PilV and β2AR.

Such a fusion protein according to the invention may for example comprise or consist of:
a meningococcal PilE protein or an immunogenic fragment of at least 10 consecutive amino acids thereof, fused in frame with a meningococcal PilV protein or an immunogenic fragment of at least 10 consecutive amino acids thereof; or
a meningococcal PilE protein or an immunogenic fragment of at least 10 consecutive amino acids thereof, fused in frame with a meningococcal vaccine antigen; or
a meningococcal PilV protein or an immunogenic fragment of at least 10 consecutive amino acids thereof, fused in frame with a meningococcal vaccine antigen; or
a meningococcal PilE protein or an immunogenic fragment of at least 10 consecutive amino acids thereof, fused in frame with (i) a meningococcal PilV protein or an immunogenic fragment of at least 10 consecutive amino acids thereof, and (ii) a meningococcal vaccine antigen.

For example, said fusion protein comprises or consists of the immunogenic fragment of PilE of sequence SEQ ID NO: 18 fused with the fHBP polypeptide of sequence SEQ ID NO: 23, or of the immunogenic fragment of PilE of sequence SEQ ID NO: 20 fused with the fHBP polypeptide of sequence SEQ ID NO: 23. Consequently said fusion protein comprises or consists of the sequence SEQ ID NO: 27 or SEQ ID NO: 28.

In another preferred embodiment, said combination comprises or consists of the immunogenic fragment of PilV of sequence SEQ ID NO: 19 fused with the fHBP polypeptide of sequence SEQ ID NO: 23, or of the immunogenic fragment of PilV of sequence SEQ ID NO: 21 fused with the fHBP polypeptide of sequence SEQ ID NO: 23. Consequently said fusion protein comprises or consists of the sequence SEQ ID NO: 29 or SEQ ID NO: 30.

The fusion protein according to the invention may further comprise other moieties such as e.g. peptidic linkers allowing linking the different antigens, proteins or fragments comprised within the fusion protein, a tag useful for purification, a leader sequence, a signal peptide, one or more peptidic fragments enhancing the immunogenicity, etc.

The invention further provides:
the above fusion proteins for use as a vaccine for preventing a meningococcal infection, in particular for preventing the spreading of meningococci into the meningeal space;
nucleic acids encoding the above fusion proteins;
vaccine compositions that comprise the above fusion proteins or nucleic acids together with a pharmaceutically acceptable carrier.

The invention also relates to a vaccine composition or immunogenic composition comprising or consisting of:

a meningococcal PilE or PilV protein, or an immunogenic fragment of at least 10 consecutive amino acids thereof according to the invention,

a combination according to the invention, or

a fusion protein according to the invention.

More particularly, said vaccine composition or immunogenic composition comprises or consists of a combination comprising or consisting of at least two of:
a meningococcal PilE protein, or an immunogenic fragment of at least 10 consecutive amino acids thereof,
a meningococcal PilV protein, or an immunogenic fragment of at least 10 consecutive amino acids thereof; and
a meningococcal vaccine antigen,
   wherein said meningococcal vaccine antigen is not PilE or PilV or an immunogenic fragment thereof.

For example, said vaccine composition or immunogenic composition comprises or consists of a combination comprising or consisting of the immunogenic fragment of PilE of sequence SEQ ID NO: 18 and the fHBP polypeptide of sequence SEQ ID NO: 23, or of the immunogenic fragment of PilE of sequence SEQ ID NO: 20 and the fHBP polypeptide of sequence SEQ ID NO: 23.

For example, said vaccine composition or immunogenic composition comprises or consists of a combination comprising or consisting of the immunogenic fragment of PilV of sequence SEQ ID NO: 19 and the fHBP polypeptide of sequence SEQ ID NO: 23, or of the immunogenic fragment of PilV of sequence SEQ ID NO: 21 and the fHBP polypeptide of sequence SEQ ID NO: 23.

Still more particularly, said vaccine composition or immunogenic composition comprises or consists of a fusion protein comprising or consisting of at least two of:
a meningococcal PilE protein, or an immunogenic fragment of at least 10 consecutive amino acids thereof;
a meningococcal PilV protein, or an immunogenic fragment of at least 10 consecutive amino acids thereof; and
one or more meningococcal vaccine antigen(s),
   wherein said one or more meningococcal vaccine antigen(s) is not PilE or PilV or an immunogenic fragment thereof.

For example, said vaccine composition or immunogenic composition comprises or consists of a fusion protein comprising or consisting of the immunogenic fragment of PilE of sequence SEQ ID NO: 18 fused with the fHBP polypeptide of sequence SEQ ID NO: 23, or of the immunogenic fragment of PilE of sequence SEQ ID NO: 20 fused with the fHBP polypeptide of sequence SEQ ID NO: 23. Accordingly, said vaccine composition or immunogenic composition comprises or consists of a fusion protein comprising or consisting of the sequence SEQ ID NO: 27 or SEQ ID NO: 28.

For example, said vaccine composition or immunogenic composition comprises or consists of a fusion protein comprising or consisting of the immunogenic fragment of PilV of sequence SEQ ID NO: 19 fused with the fHBP polypeptide of sequence SEQ ID NO: 23, or of the immunogenic fragment of PilV of sequence SEQ ID NO: 21 fused with the fHBP polypeptide of sequence SEQ ID NO: 23. Accordingly, said vaccine composition or immunogenic composition comprises or consists of a fusion protein comprising or consisting of the sequence SEQ ID NO: 29 or SEQ ID NO: 30.

The invention also provides the use of a vaccine composition or an immunogenic composition according to the invention for use for preventing meningococcal infection, or for preventing meningococci to cross of the BBB or to spread into the meningeal space.

Methods of obtaining a vaccine composition or immunogenic composition are well known in the art. Generally such methods involve extracting proteins from bacterial preparations using techniques such as sonication, proteolytic digestion, heat treatment, freeze-thaw treatment, osmotic shock treatment etc. . . . . Examples of artificial bacterial preparations include protein preparations either in part or entirely obtained by synthetic or recombinant methods. Oligonucleotides can probes can be devised based on the sequences of the bacterial genome and can be used to probe genomic or cDNA libraries for genes encoding antigens useful in the context of the invention. Genes can be isolated using standard techniques.

The vaccine composition or immunogenic composition can comprise a suitable adjuvant. The type of adjuvant will vary, depending on the type of antigen preparation and route of administration used. Any adjuvant known in the art may be used in the immunogenic composition, including oil-based adjuvants such as Freund's Complete Adjuvant and Freund's Incomplete Adjuvant, mycolate-based adjuvants (e.g., trehalose dimycolate), bacterial lipopolysaccharides (LPS), peptidoglycans (i.e., mureins, mucopeptides, or glycoproteins such as N-Opaca, muramyl dipeptide [MDP], or MDP analogs), proteoglycans (e.g., extracted from Klebsiella pneumoniae), streptococcal preparations (e.g., OK432), Biostim™. (e.g., 01K2), the “iscorns” of EP 109 942, EP 180 564 and EP 231 039, aluminum hydroxide, saponin, DEAE-dextran, neutral oils (such as miglyol), vegetable oils (such as arachis oil), liposomes, Pluronic® polyols. Adjuvants include, but are not limited to, the RIBI adjuvant system (Ribi Inc.), alum, aluminum hydroxide gel, cholesterol, oil-in water emulsions, water-in-oil emulsions such as, e.g., Freund's complete and incomplete adjuvants, Block co-polymer (CytRx, Atlanta Ga.), SAF-M (Chiron, Emeryville Calif.), AMPHIGEN® adjuvant, saponin, Quil A, QS-21 (Cambridge Biotech Inc., Cambridge Mass.), GPl0100 (Galenica Pharmaceuticals, Inc., Birmingham, Ala.) or other saponin fractions, monophosphoryl lipid A, Avridine lipid-amine adjuvant, heat-labile enterotoxin from E. coli (recombinant or otherwise), cholera toxin, or muramyl dipeptide, among many others.

More particularly, said vaccine composition or immunogenic composition raises an immune response with antibodies that prevent meningococcal infection, or that prevent meningococci to cross of the BBB or to spread into the meningeal space. Still more particularly, said vaccine or vaccine compositions induce the production of protective antibodies inhibiting the interaction between PilE and/or PilV and β2AR.

Suitable pharmaceutically acceptable carrier is for example an adjuvant. Adjuvants include, but are not limited to oil-based adjuvants such as Freund's Complete Adjuvant and Freund's Incomplete Adjuvant, mycolate-based adjuvants (e.g., trehalose dimycolate), bacterial lipopolysaccharides (LPS), peptidoglycans (i.e., mureins, mucopeptides, or glycoproteins such as N-Opara, muramyl dipeptide [MDP], or MDP analogs), proteoglycans (e.g., extracted from Klebsiella pneumoniae), streptococcal preparations (e.g., OK432), Biostim™ (e.g., 01K2), the “Iscoms” of EP 109 942, EP 180 564 and EP 231 039, aluminum hydroxide, saponin, DEAE-dextran, neutral oils (such as miglyol), vegetable oils (such as arachis oil), liposomes, Pluronic™ polyols, the RlBl adjuvant system (Ribi Inc.), alum, aluminum hydroxide gel, cholesterol, oil-in water emulsions, water-in-oil emulsions such as, e.g., Freund's complete and incomplete adjuvants, Block co-polymer (CytRx, Atlanta Ga.), SAF-M (Chiron, Emeryville Calif), AMPHIGEN™ adjuvant, saponin, Quil A, QS-21 (Cambridge Biotech Inc., Cambridge Mass.), GPI-0100 (Gaienica Pharmaceuticals, Inc., Birmingham, Ala.) or other saponin fractions, monophosphoryl lipid A, Avridine lipid-amine adjuvant, heat-labile enterotoxin from E. coli (recombinant or otherwise), cholera toxin, or murarnyi dipeptide, among many others.

The present invention also provides a method for preventing meningococcal infection in a subject comprising the step of administering to a subject in need thereof an effective amount of a vaccine composition, wherein said vaccine composition comprises:
a meningococcal PilE or PilV protein, or an immunogenic fragment of at least 10 consecutive amino acids thereof, or
the combination according to the invention, or
the fusion proteins acording to the invention

In a preferred embodiment, the invention provides a method for preventing meningococcal infection in a subject comprising the step of administering to a subject in need thereof an effective amount of a meningococcal PilE or PilV protein, or an immunogenic fragment of at least 10 consecutive amino acids thereof.

In another preferred embodiment, the invention provides a method for preventing meningococcal infection in a subject comprising the step of administering to a subject in need thereof an effective amount of combination of at least two of:
a meningococcal PilE protein, or an immunogenic fragment of at least 10 consecutive amino acids thereof;
a meningococcal PilV protein, or an immunogenic fragment of at least 10 consecutive amino acids thereof; and
one or more meningococcal vaccine antigen(s),
   wherein said one or more meningococcal vaccine antigen(s) is not PilE or PilV or an immunogenic fragment thereof.

In still another preferred embodiment, the invention provides a method for preventing meningococcal infection in a subject comprising the step of administering to a subject in need thereof an effective amount of combination of at least two of:
a meningococcal PilE protein, or an immunogenic fragment of at least 10 consecutive amino acids thereof;
a meningococcal PilV protein, or an immunogenic fragment of at least 10 consecutive amino acids thereof; and
one or more meningococcal vaccine antigen(s),
   wherein said at least two proteins, fragments or antigens are administered simultaneously or sequentially.

In still another preferred embodiment, the invention provides a method for preventing meningococcal infection in a subject comprising the step of administering to a subject in need thereof an effective amount of fusion proteins comprising at least two of:
a meningococcal PilE protein, or an immunogenic fragment of at least 10 consecutive amino acids thereof;
a meningococcal PilV protein, or an immunogenic fragment of at least 10 consecutive amino acids thereof; and
one or more meningococcal vaccine antigen(s).

In still another preferred embodiment, the invention provides a method for preventing meningococcal infection in a subject comprising the step of administering to a subject in need thereof an effective amount of an immunogenic composition comprising or consisting of:
a PilE protein, or a fragment of at least 10 consecutive amino acids thereof, and/or
a PilV protein, or a fragment of at least 10 consecutive amino acids thereof, and
a meningococcal vaccine antigen.

Preferably, said meningococcal vaccine antigen is not a PilE or pilV protein, or fragment thereof.

As used herein, “an effective amount” means that the administered component or components are in an amount that is sufficient to induce a specific immune response against a meningococcus in the subject being vaccinated. The effective amount can be readily determined by one skilled in the art, for example, through conventional experiments on animals.

The immunogenic composition, the vaccines and the fusion proteins according to the invention can be administered via any suitable route, such as by mucosal (intranasal), parenteral, or intramuscular administration, oral, intradermal, intraperitoneal, intravenous, or subcutaneous administration. Suitable vaccination routes also comprise combination administrations (e.g. oral/intramuscular administration).

The immunogenic composition, the vaccines and the fusion proteins according to the invention can be administered alone or with suitable pharmaceutical carriers, and can be in solid or liquid form such as, tablets, capsules, powders, solutions, suspensions, or emulsions.

The solid unit dosage forms can be of the conventional type. The solid form can be a capsule, such as an ordinary gelatin type containing the proteins or peptides of the present invention or the antibodies or binding portions thereof of the present invention and a carrier, for example, lubricants and inert fillers such as, lactose, sucrose, or cornstarch. In another embodiment, these compounds are tableted with conventional tablet bases such as lactose, sucrose, or corn starch in combination with binders like acacia, corn starch, or gelatin, disintegrating agents such as, corn starch, potato starch, or alginic acid, and a lubricant like stearic acid or magnesium stearate.

The immunogenic composition, the vaccines and the fusion proteins according to the invention may also be administered in injectable dosages by solution or suspension of these materials in a physiologically acceptable diluent with a pharmaceutical carrier. Such carriers include sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions.

For use as aerosols, the immunogenic composition, the vaccines and the fusion proteins according to the invention of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

All references cited herein, including journal articles or abstracts, published patent applications, issued patents or any other references, are entirely incorporated by reference herein, including all data, tables, figures and text presented in the cited references.

Although having distinct meanings, the terms “comprising”, “having”, “containing” and “consisting of” have been used interchangeably throughout this specification and may be replaced with one another.

In the frame of the present application, all agents, molecules, compounds, proteins, polypeptides, peptides, nucleic acids, vectors and host cells may be isolated and/or purified.

The invention will be further evaluated in view of the following examples and figures.

### BRIEF DESCRIPTION OF THE FIGURES

FIG. 1. Requirement of Grk2 and βArrs for Nm-Induced Ezrin Honeycombs.

Left panel: Effect of siRNA-dependent inhibition of βarrs, GRK2, and Gαₛ expression on ezrin recruitment under Nm colonies; the recruitment assay is described in Experimental Procedures. Data are the average values (±SEM) of ezrin recruitment compared to the control (scramble siRNA-treated cells) from 3 independent experiments in triplicate. ***(p<0.001). Right panel: Effect of siRNA-dependent inhibition of β2AR and GRK2 expression on βarr2 recruitment under Nm colonies; the recruitment assay is as in (c) except that the readout is based on βarr2-GFP accumulation.

FIG. 2. β2AR Involvement in Ezrin Recruitment to Nm Colonies.

Top left panel; Effect of pre-treatment with saturating concentrations of specific GPCR agonists on Nm-induced ezrin recruitment in hCMEC/D3 cells. ISO: β2AR agonist isoproterenol, 5 μM; SDF-1: CXCR4 agonist, 100 nM; MlP-1β: CCRS agonist, 100 nM; IL-8: CXCR1-2 agonist, 100 nm; Ang II; angiotensin II, ATIR agonist, 10 nM. ***(p<0.001). Top right panel: Dose-dependent effect of isoproterenol on Nm-promoted ezrin recruitment. Bottom left panel: Effect of endocytosis inhibitors on isoproterenol modulation of Nm-promoted ezrin recruitment, hCMEC/D3 cells were transfected with plasmids for either DIII (dominant negative mutant of Eps15 fused to GFP) or βarrC-ter (carboxyterminal tail of βarr fused to GFP), 24 h before isoproterenol pretreatment (+) and subsequent ezrin recruitment assay; (−) control cells pre-treated with medium alone; ***(p<0.001), Bottom right panel: Reversal of the effect of isoproterenol pre-treatment on Nm-promoted ezrin recruitment by propranolol and inhibition of ezrin honeycombs by pre-treating hCMEC/D3 cells with β2AR specific siRNA. ISO: 10 μM isoproterenol, Propra: 100 nM propranolol; ***(p<0.001)

FIG. 3. Reconstitution of Nm-Promoted Signaling in HEK-293 Cells

Ezrin recruitment assay in HEK-293 cells transfected with the indicated plasmids and infected with the indicated Nm strains. AT1R: control Type 1 Ang II receptor. Bars indicate SEM from 3 experiments in duplicate. ***p<0.001. Other control receptors were tested (CCR5, CXCR4), which all failed to reconstitute ezrin recruitment above the values observed for AT1R (not shown).

FIG. 4. Effect of Isoproterenol on Nm Adhesion and Growth Under Flow

Left panel: “Under flow” adhesion of bacteria on hCMEC/D3 cells pre-treated with 10 μM isoproterenol or felt untreated. Adhesion under shear stress was determined by infecting cells grown in IBIDI™ flow chamber under a shear stress of 0.04 dyn/cm² as described in Experimental Procedures. No significant difference was observed for static adhesion in the absence or presence of isoproterenol (p=0.73, n=6), whereas after pre-treatment with the β2AR agonist, “under flow” studies showed a 40% reduction of bacterial adhesion (*p=0.018, n=6). Right panel: Analysis of the size of Nm colonies growing on hCMEC/D3 cells under flow in control conditions or after cell pretreatment with 10 μM isoproterenol or forskolin for 1 h before injection of bacteria. Colonies were classified in 3 groups according to their size for each indicated time (see Experimental Procedures for details).

FIG. 5. Nm Crossing of hCMEC/D3 Cell Monolayers.

Left panel: Transmigration assays of Nm on hCMEC/D3 cells grown on 3 μm pore size MilliCell® inserts. Transmigration in the presence or absence of isoproterenol pre-treatment (1h, 10 μM) was expressed as the number of migrating bacteria through the cell monolayer per hour. Data correspond to 5 independent experiments in duplicate; ***p<0.001. Right panel: Surface and number of cell-cell junction gaps induced by Nm, with or without isoproterenol pretreatment (10 μM, 1 h), visualized by confocal fluorescence microscopy on fixed MilliCell® inserts immunostained for VE-cadherin. The areas of gaps (see experimental procedures) were classified in 3 groups according to the number of bacteria that can be accommodated in the gap: 4 diplococci or less, 4 to 8 diplococci, more than 8 diplococci ***p<0.001, **p<0.01. The total surface occupied by bacteria on the apical surface of the monolayer, determined by anti-pili staining, was similar with or without isoproterenol (not shown).

FIG. 6. Role of βarrs in Nm-Promoted Signaling.

Top panels: Docking and activation of Src. Top left panel: Reduction of Src recruitment under Nm colonies in hCMEC/D3 cells treated with β2AR or βarr(1+2) siRNAs; n=6, **p<0.01 Top middle panel: Dominant negative effect of βarr2-Src-GFP on actin recruitment in the same cells. Details on βarr2-ΔSrc construct are in Supplemental Methods. n=6, ***p<0.001 Top right panel: Size of Nm colonies growing on hCMEC/D3 cell monolayers under laminar flow in control GFP-transfected cells, and in cells expressing either barr2-GFP or dominant negative βarr2-ΔSrc-GFP. Bottom panel: βarr-dependent recruitment of junctional proteins into cortical plaques. Effect of the siRNA-mediated inhibition of βarr1 and βarr2 gene expression on junctional and cytoskeletal protein recruitment into cortical plaques in Nm-infected hCMEC/D3 cells. Data correspond to average values (±SEM) of 3 independent experiments in duplicate. ***p<0.001, **p<0.01 relative to control.

FIG. 7. Molecular Determinants of Nm-β2AR Interaction

Top left panel: Ezrin recruitment assay in wt or β2AR− and βarr-reconstituted Hek-293 cells (as in FIG. 3) infected with a piliated SiaD− Opa+ Nm strain displaying normal or PilV− pili or with a non-piliated SiaD− Opa+ strain (PilE−); ***p<0.001. To right panel: The upper left cartoon depicts the approach to examine the respective role of pilins. Receptor recruitment was counted in all cells in contact with staphylococci aggregates; data correspond to average values (±SEM) of 3 independent experiments in duplicate. ***p<0.001, **p<0.01. Bottom panel: Ezrin recruitment experiment comparing the β2AR the AT1R and the chimeric Nterβ2AR-AT1R: ***p<0.001 compared to control and AT1R.

### BRIEF DESCRIPTION OF THE SEQUENCE LISTING

SEQ ID NO: 1 shows the amino acid sequence of PilE.
SEQ ID NO: 2 shows the amino acid sequence of PilV.
SEQ ID NO: 3 shows the amino acid sequence of β2AR. The position of the N-terminus present in the Nterβ2AR-AT1R construct is indicated.
SEQ ID Nos. 4 to 7 show the sequences of siRNAs used in Example 1.9.
SEQ ID Nos, 8 to 11 show the sequences of primers used in Example 1.16.
SEQ ID Nos. 12 to 15 show the sequences of primers used in Example 1.18.
SEQ ID NO: 16 shows the sequence of the hydrophobic domain of PilE.
SEQ ID NO: 17 shows the sequence of the hydrophobic domain of PilV.
SEQ ID NOs: 18 and 20 show the sequences of immunogenic fragments of PilE,
SEQ ID NOs: 19 and 21 show the sequences of immunogenic fragments of PilV.
SEQ ID NOs: 22 and 23 show the sequences of the fHBP polypeptides.
SEQ ID NO: 24 shows the sequence of the PorA polypeptide.
SEQ ID NO: 25 shows the sequence of the NHBA polypeptide.
SEQ ID NO: 26 shows the sequence of the NadA polypeptide.
SEQ ID NOs: 27 and 28 show the sequences of the fusion proteins between PilE and fHBP polypeptide.
SEQ ID NOs: 29 and 30 shows the sequences of the fusion proteins between PilV and fHBP polypeptide.

### EXAMPLES

### Example 1

### Protocols and Experimental Procedures

### 1.1. Cell Line and Bacterial Strains

The hCMEC/D3 cell line, retaining the main characteristics of primary brain endothelial cells, was described previously (Weksler et al., 2005, FASEB J 19, 1872-1874). A piliated capsulated Opa variant of serogroup-C meningococcal strain 8013, designated 2C43 (Nassif et al., 1993. Mol Microbiol 8, 719-725), was used throughout the study unless otherwise specified. Non-capsulated isogenic derivatives were engineered by introducing a siaD mutation. Isogenic non-piliated, piliated or PilV− variants of an Opa⁺ non-capsulated 2C43 derivative were designated SiaD− Opa⁺ PilE, SiaD− Opa⁺ and SiaD− Opa⁺ PilV respectively. Nm expressing the green fluorescent protein (GFP) were obtained by introducing in bacteria the pAM239 plasmid by conjugation (Mairey et al., 2006, J Exp Med 203, 1939-1950).

### 1.2. Quantitative Analysis of Protein Recruitment Under Bacterial Colonies

The recruitment efficiency was estimated by determining the proportion of colonies positive for the indicated protein of interest. At least 50 colonies were observed per coverslip. Each experiment was repeated several times in duplicate or triplicate. Data was examined for significance using Student's t-test.

### 1.3. Transfections

hCMEC/D3 cells were transfected using the Amaxa Nucleofactor Kit for Huvec (Amaxa Biosystem) or jetPEl transfection reagent (Polyplus Transfection) according to the manufacturer's instructions. HEK-293 cells were transfected using Lipofectamine 2000 (Invitrogen).

### 1.4. Nm Adhesion Under Shear Stress

hCMEC/D3 cells were grown on disposable flow chambers (15 μ-Slide VI, IBIDI) coated with 5 μg/cm² of rat-tail collagen type I. For experiments investigating Nm colony growth under shear stress, 10⁷ bacteria were introduced into the flow chamber and allowed to adhere for 10 min without flow. Subsequently, a flow corresponding to a shear stress of 0.2 dyn/cm² was applied using a syringe pump (Harvard Apparatus). For adhesion assays under shear stress, 10⁷ bacteria were allowed to adhere for 20 min under a shear stress of 0.04 dyn/cm², then the chambers were washed 1 min under a shear stress of 1 dyn/cm² (Mairey et al., 2008. J Exp Med 203, 1939-1950).

### 1.5. Nm Transmigration Assay

hCMEC/D3 cells were grown on 3 μm pore size MilliCell® inserts and infected at a MOI of 10, corresponding to 10⁶ bacteria per insert. Four hours after infection, inserts were placed in a well with fresh medium. After additional 30 min, the number of bacteria, which had crossed the cell monolayer was determined by plating the content of the well and counting the number of colony forming units. To estimate the surface and number of macroscopic cell-cell junction openings, MilliCell® inserts were fixed in 4% PFA and immunostained for VE-cadherin and actin, Several baso-lateral cross-sections of each insert were acquired using a Leica SP5 confocal microscope. Surface and number of gaps within the hCMEC/D3 cell monolayer was determined using Leica Application Suite-AF (LAS-AF) software. Data were expressed as the number of gaps per 4×10 ⁵ μm² of hCMEC/D3 cell monolayer surface. The size of gaps was determined using the apparent size of a Nm diplococcus as surface unit (approximately 1.7 μm²).

### 1.6. Cell Culture

The immortalized human brain micro-vascular endothelial cell line hCMEC/D3, which retains the main characteristics of primary brain endothelial cells, was described previously. hCMEC/D3 cells were grown at a density of 25×1 03 cells per cm² in flasks coated with 5 μg/cm 2 rat tail collagen type I (BD Bioscience), in EBM-2 medium (Lonza) supplemented with 5% of Fetal Calf Serum, 1% of Penicillin-Streptomycin, 1.4 μM hydrocortisone, 5 μg/ml ascorbic acid, 10 mM HEPES and 1 ng/ml b-FGF, at 37° C. in a humidified incubator in 5% CO2. Human Embryonic Kidney 293 (HEK-293) cells were grown in flasks coated with 0.01% poly-L-lysine (Sigma), containing DMEM medium supplemented with 10% Fetal Calf Serum and 1% Penicillin-Streptomycin, in 5% CO2-enriched humidified atmosphere. For immunofluorescence assays, cells were grown on 12-mm diameter glass coverslips with appropriate coating and media. The ability of bacteria to transmigrate through hCMEC/D3 cells monolayers was assayed with Millicell® inserts of indicated pore size. Cells were seeded at a density of 5×1 04 cells per cm2 on inserts pre-coated with rat tail collagen type I (thick coating, modified from Bornstein and Murray) for 12-15 days. The medium was replaced every 3-4 days and 24 h before the experiment.

### 1.7 Bacterial Strains, Growth and Infection Conditions

A piliated capsulated Opa− variant of serogroup-C meningococcal strain 8013, designated 2C43, was used throughout the study (Nassif et al., 1993, Mol Microbiol 8, 719-725). A non-capsulated derivative of this strain was engineered by introduction of a siaD mutation. Isogenic non-piliated and piliated Opa⁺ strains of this non-encapsulated 2C43 derivative were designated SiaD− Opa⁺ PilE− and SiaD− Opa⁺, respectively (Coureuil et al., 2009, Science 325, 83-87). Isogenic piliated and non-capsulated Opa+ mutant for the pilV gene was designated SiaD− Opa+ PilV− (Mikaty et al., 2009, PLoS Pathog 5, e1000314). N. meningitidis expressing the green fluorescent protein (GFP) were obtained by introducing in bacteria the pAM239 plasmid by conjugation (Mairey et al., 2006, J Exp Med 203, 1939-1950). The day before the infection, hCMEC/D3 cells were serum-starved in EBM-2 medium containing 0.1% Bovine Serum albumin (BSA). The day of infection, bacteria from an overnight culture were adjusted to OD600=0.05 and incubated for 2 h at 37° C. in pre-warmed starvation medium. Bacteria were added to cells at a multiplicity of infection (MOI) of 100 and allowed to adhere for 30 minutes. Unbound bacteria were washed with fresh medium and infection was allowed to proceed for 2 h. When needed, drugs were added to cell culture 1 h before infection. Bacteria were tested for growth and aggregation. Bacterial colonies harvested from GCB agar plates were grown to OD600=0.05 in EBM2, 0.1% BSA in the presence or Absence of 100 μM isoproterenol and incubated at 37° C. in a 5% CO2 atmosphere under constant shaking. The OD600 was then measured after 1, 2, 3, 4 and 8 h. Aggregation was determined after 2 h of incubation in EBM2, 0.1% BSA in the presence or absence of 100 μM isoproterenol at 37° C. in a 5% CO2 atmosphere under constant shaking. A cone and plate assay was then performed as described previously (Mikaty et al., 2009) using a cone and plate rheometer (Brookfield Engineering Laboratories Inc., Middleboro, Mass., USA).

### 1.8. Plasmids and Transfections

Plasmids coding for βarr1-GFP, βarr2-GFP, myc-βarr1 and myc-βarr2, the carboxyterminal tail of βarr2-fused to GFP, the human β2AR-YFP (β2AR-YFP) the AT1R, CCR5 and CXCR4 were described previously, as were plasmids coding for dominant negative Eps15 (Eps15-DIII-GFP) and control Eps15-DIIIΔ2-GFP, and the plasmid encoding CEACAM-1. The 4×CRE-Luciferase reporter plasmid was purchased from Stratagene. βarr2-ΔSrc (βarr2 P91G P93D, P94R, P97L) was prepared by site directed mutagenesis using βarr2-GFP or myc-βarr2 as templates and the QuikChange (Stratagene) protocol.

The chimeric Nterβ2AR-AT1R receptor was constructed using a 2-step procedure. First a unique Kpn1 site was introduced by site-directed mutagenesis in the human AT1R immediately before the beginning of the first transmembrane domain (position F28). The N-terminus of the human β2AR (amino acids 1 to 28 of SEQ ID NO: 3), amplified by PCR, was then subcloned in phase upstream of the codon for F28 using the Kpn1 site. The constructs for β2AR-ΔSrc and Nterβ2AR-AT1R were sequenced to verify the phases and the absence of undesired mutations (DNA sequence facility, Institut Cochin). hCMEC/D3 cells were transfected using the Amaxa Nucleofactor Kit for Huvec (Amaxa Biosystem) or jetPEI transfection reagent (Polyplus Transtection) according to manufacturer instructions. HEK-293 cells were transfected using Lipofectamine 2000 (Invitrogen) following manufacturer instructions.

### 1.9. Inhibition of Protein Expression by ANA Interference

Small interfering RNAs targeting human βarrestin-1 (5′-AAAGCCUUCUGCGCGGAGAAU-3′. SEQ ID NO: 4), βarrestin-2 (5′-GGCUUGUCCUUCCGCAAAGAC-3′, SEQ ID NO: 5), and GRK2 (5′-AAGAAGUACGAGAAGCUGGAGUU-3′, SEQ ID NO: 6) were synthesized by Eurogentech. To silence the expression of Gas and of the β2-adrenoceptor, a pool of four siRNA duplexes (ON-Target plus SMARTpool, NM_080426 and NM_000024 from Dharmacon, respectively) were used. The efficacy of knockdown was assessed three days after transfection by immunoblotting or by RT-PCR assays using whole cell lysates. An offtarget siRNA (5′-GATAAGGTGCTGCGTGGACCC-3′, SEQ ID NO: 7) and the siCONTROL siRNA (Dharmacon) were used as controls. Si RNAs were transfected using Amaxa Nucleofactor Kit for Huvec according to manufacturer's instructions. The infections with Nm were performed three days after transfection.

### 1.10. Antibodies, Drugs and Receptor Ligands

The following monoclonal antibodies (mAb) or polyclonal antibodies were used for immunofluorescence labeling or immunoblotting: anti-ezrin rabbit antibody, anti-βarr rabbit A1CT antibody, rabbit monoclonal anti-βarr2 antibody (C16D9, Cell Signaling), anti-Gαₛ rabbit antibody, anti-GRK2 mouse mAb (clone C5/1.1, Upstate), anti-src rabbit polyclonal antibody (SC-18, Santa Cruz), anti-phosphorylated Src (PY418) rabbit monoclonal antibody (Bioscience, #44-660G), anti-actin rabbit polyclonal antibody (Sigma-Aldrich), anti-VE-cadherin rabbit polyclonal antibody (Bender Medsystems), anti-p120-catenin mouse mAb (BD Transduction Laboratories), anti-myc mouse mAb or rabbit polyclonal anti body (Cell Signaling), anti -type IV pili mouse mAb 20D9 and anti-GFP mouse mAb 3E6 (Invitrogen). Following Goat secondary antibodies were used for Immunofluorescence labelling or immunoblotting: anti-mouse IgG (H+L) and anti-rabbit IgG (H+L) coupled to Horse Radish Peroxidase (Jackson ImmunoResearch Laboratories) and anti-mouse IgG (H+L) and anti-rabbit IgG (H₊L) coupled to Alexa-488 or Alexa-546 or Alexa-633 (Invitrogen). Isoproterenol, propranolol, angiotensin II (Ang II) forskolin and PP2 Src inhibitor were purchased from Sigma-Aldrich. SDF-1 (agonist of CXCR4 receptor), MIP-1β (agonist of CCR5 receptor), IL8 were all from Preprotech.

### 1.11. Immunofluorescence Experiments

Cells were fixed for 20 min in PBS 4% PFA and permeabilized for 5 min in PBS containing 0.1% Triton X-100. Cells were incubated with primary antibodies at the recommended concentration in PBS containing 03% BSA for 1 h, After 3 washings in the same buffer, Alexa-conjugated phalloidin and DAPI (0.5 μg/ml) were added to Alexa-conjugated secondary antibodies for 1 h. After additional washings, coverslips were mounted in mowiol. Image acquisition was performed on a laser-scanning confocal microscope (Zeiss LSM 510). Images were collected and processed using the Leica Application Suite AF lite (Leica) software. 3D reconstruction was performed using the IMARIS software.

### 1.12 Quantitative Analysis of Ezrin (and Other Proteins) Recruitment Under Bacterial Colonies

After infection, cells were treated for immunofluorescence analysis with appropriate antibodies and observed under a light fluorescent microscope. For each Nm strain, the recruitment efficiency was estimated by determining the proportion of colonies that efficiently recruited the protein of interest in a “honeycomb shape” just under the colonies. Importantly, in uninfected cells, no ezrin, actin or Src were found in such honeycomb-shaped clusters at the plasma membrane. At least 50 colonies were scrutinized per coverslip. Each experiment was repeated several times (at least 3). Data was examined for significance using Student's t-test.

### 1.13. CRE-Lucifetase Assay

Luciferase activity in cell lysates was measured using the Luciferase Reporter Assay System (Promega) according to manufacturer instructions. Briefly, hCMEC/D3 cells were transfected with the 4×CRE-Luciferase plasmid (Stratagene) the day before the experiment. Cell infection was performed as described above at a MOI of 100. After 2 h of infection or incubation with drugs or ligands, cells were washed twice with ice-cold PBS, and lysed using the Luciferase Cell Culture Lysis Reagent. Lysates were centrifuged at 12,000×g for 2 min at 4° C. Supernatants were tested for luciferase expression using a V ICTOR 3 multilabel counter (Perkin-Elmer).

### 1.14 Nm Adhesion Under Sheer Stress

Shear stress assays were performed as described previously (Mairey et al., 2006, J Exp Med 203, 1939-1950). Briefly, cells were grown on disposable flow chambers (15 μ-Slide VI, IBIDI) coated with 5 μg/cm2 rat tail collagen type I. Bacteria grown to OD600=0.05 in EBM2, 0.1% BSA medium were incubated for 2 h at 37° C. under 5% CO2 atmosphere. GFP expression in bacteria was induced by adding 1 mM IPTG in EBM2, 0.1% BSA. For experiments aimed at determining the growth of colonies under shear stress, 107 bacteria were then introduced into the flow chamber and allowed to adhere for 20 min without flow. Subsequently, a flow corresponding to a shear stress of 0.2 dyn/cm2 was applied using a syringe pump (Harvard Apparatus). For adhesion assays under shear stress, 10 bacteria were introduced into the flow chamber and allowed to adhere for 20 min under a shear stress of 0.04 dyn/cm2, subsequently the chamber was washed 1 min under a shear stress of 1 dyn/cm2, Fluorescent bacteria in the chambers were observed using an Olympus CKX41 inverted microscope with a 20× objective, equipped with a shutter for the fluorescence I amp and a Hamamatsu ORCA285 CCD camera. Images were collected and processed using the Openlab software (Improvision). The field under observation corresponded to a surface of 425 μm by 320 μm with a resolution of 0.63 μm per pixel. Images were analyzed using the lmageJ software.

### 1.15. Nm Transmigration Assay

Cell permeability to Nm was determined as described previously (Coureuil et al., 2009, Science 325, 83-87). Briefly, hCMEC/D3 cells were grown on 3 μm pore size MilliCell® inserts and infected at a MOI of 10 (10⁶ bacteria per insert). Four hours after infection, inserts were placed in a well with fresh medium. After 30 min, the number of bacteria, which had crossed the monolayer of endothelial cells, was then measured by plating the content of dilutions of the lower compartment on agar plates and counting the number of colony forming units (CFU). The effect of isoproterenol on Nm transmigration was determined by comparing the number of migrating bacteria through the monolayer of endothelial cells per hour in the presence and absence of the agonist. To estimate the surface and number of macroscopic cell-cell junction openings, MilloCell ® inserts were fixed at the end of the experiments in 4% PFA and immunostained for V E-cadherin and actin. Several baso-lateral cross-sections of each insert were acquired using a Leica SP5 confocal microscope. The surface and number of gaps within the hCMEC/D3 cell monolayer was determined using the Leica Application Suite-AF (LAS-AF) software. Data were expressed as the number of gaps per 4.10⁵ μm² of hCMEC/D3 cell monolayer surface. The size of gaps was determined using the size of a Nm diplococcus as surface unit (approximately 1.7 μm²). The total surface occupied by the colonies was determined using an anti-pili staining and analyzed using ImageJ software. This total surface was similar with or without isoproterenol.

### 1.16. Expression and Purification of MBP-Pilin Recombinant Proteins

Recombinant MBP-pilin fusion proteins were produced as described before (Helaine, 2007, Proc Natl Acad Sci USA. 104,15888-15893). Fragments of pilV and comP genes lacking the region coding for the amino acid residues 1 to 28 of the full-length proteins were amplified by PCR and subcloned in pMA Lp2X vector (New England Biolabs). The resulting plasmids, pMAL-pilV and pMAL-comP, contain in frame the malE gene, which encodes the maltose-binding protein (MBP), followed by the Factor X a protease recognition site and the truncated pilin coding regions. MBP-pilin fusion proteins are directed into E. coli periplasm, where disulfide bond formation can occur. These plasmids were transformed in the PAP5198 E. coli strain, deleted for the periplasmic enzymes: OmpT Prt and DegP. The fusion proteins were purified on amylose resin (New England Biolabs). The sequence of forward (F) and reverse (R) primers used in the PCR reactions are listed below (restriction sites used for cloning are in parentheses):

pi IVp2X F (EcoR1)
(SEQ ID NO: 8)
5′-GAATTCCGGCGCGTCCGCCTGTCGG-3′
piIVp2XR (Pst1)
(SEQ ID NO: 9)
5′-CTGCAGTTAGTCGAAGCCGGGGCAGG-3′
comPp2XF (EcoR1)
(SEQ ID NO: 10)
5′-GAATTCGAGAAAGCAAAGATAAATGC-3′
comPp2XR (Pst1)
(SEQ ID NO: 11)
5′-CTGCAGCTACTTAAATAACTTGCAGTCC-3′

1.17. Coating of Staphylococcus aureus with AMP-Pilin Fusion Proteins

S. aureus (ATCC 25923) expressing specific receptors for the Fc domain of IgG immunoglobulins were used for this experiment. Bacteria from an overnight culture were adjusted to OD600=0.05 and incubated for 2 h at 37° C. in pre-warmed LB medium. About 10⁸ bacteria were incubated with 2 μg of anti-MBP antibody for 20 min at room temperature. Bacteria were then centrifuged at 4000 rpm for 5 min, washed with warm LB 3 times, incubated with 2.5 μg of each recombinant fusion protein for 20 min at room temperature. After several washes using DMEM medium supplemented with 10% Fetal Calf Serum, bacteria were used in adhesion assays (incubations of 30 min)) or lysed in modified RIPA buffer (50 mM Tris pH 7.5, 2 mM EDTA, 150 mM NaCl, 0.1% (w/v) SDS, 0.5% (w/v) Na deoxycholate) containing 1× protease inhibitors cocktail for immunoblots (Sigma-Aldrich).

### 1.18. Reverse-PCR

To determine the efficacy of the siRNA—mediated inhibition of the β2AR, total RNA were extracted using a Rneasy Mini Kit (Qiagen) according to the manufacturer protocol. DNA contaminants were removed using Turbo DNAse (Ambion). Equal amounts of RNA from control siRNA- or β2AR siRNA-transfected cells were used as templates for reverse transcription with the Superscript II Reverse transcriptase (Invitrogen) and an oligo-dT primer, following manufacturer instructions. PCR reaction mixs was prepared containing 1×PCR Buffer (Takara), 1 μM dNTP Mix (Roche), 1 μM of each primer, 0.5 units of Takara Taq polymerase and 5 μL of cDNA. Primers used for β2AR sequence amplification were: (5′-ATTGAGACCCTGTGCGTGAT-3′, forward, SEQ ID NO: 12) and (5′-CTGAAAGACCCTGGAGTAGA-3′, reverse, SEQ ID NO: 13); primers for GAPDH sequence amplification were: (5′-TTCCTACCCCCAATGTGTCC-3′, forward, SEQ ID NO: 14) and (5′-TCTTGCTCAGTGTCCTTGCT-3′, reverse, SEQ ID NO: 15). The amplified products were subjected to electrophoresis on 1.5% agarose gels and stained with ethidium bromide.

### 1.19. Immunoprecipitation and Immunoblotting

For the experiments aimed at determining the efficacy of siRNA-dependent knockdown, whole cell lysate of hCMEC/D3 cells were washed with ice-cold PBS and lysed in modified RIPA buffer containing 1× protease inhibitors cocktail (Sigma-Aldrich). Whole cell lysates were then boiled and analyzed by SD S-PA GE. For immunoprecipitation studies, HEK-293 cells were transfected with appropriate plasmids using lipofectamine 2000. Two days after transfection dishes were lysed in immunoprecipitation buffer (10 mM Hepes, 50 mM NaCl, 5 mM EDTA, 1 mM CaCl2, 1 mM MgCl2 and 0.5% Triton X100) containing a protease inhibitor cocktail (Sigma-Aldrich), on ice for 1 h. Alter centrifugation at 13000×g for 20 min, the supernatant was collected and protein concentration determined (Bio Rad protein assay). Supernatants were incubated with the anti-myc antibody for 2 h at room temperature and then incubated with protein G-Sepharose beads for 1 h at 4° C. After centrifugation, the immunobead-bound material was washed 3 times with lysis buffer, The immunoprecipitates were resolved in 10% SDS; PAGE gels. After transfer on nitrocellulose (Optitran BA-S 83, Millipore), blots were probed with the appropriate antibodies. Immunoblots were revealed by luminescence (ECLplus; GE Healthcare Life Sciences).

### Example 2

### βarrs Play an Essential Role in the Formation of Ezrin Honeycombs Under Nm Colonies Protocols and Experimental Procedures

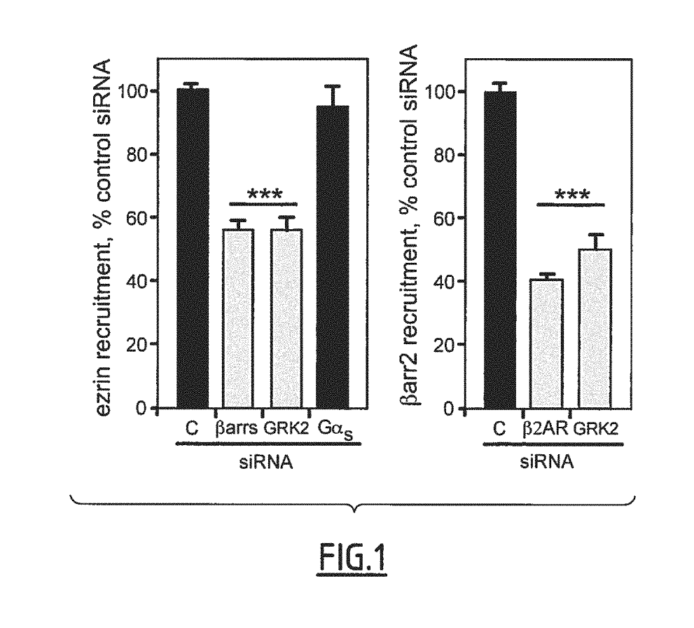

To investigate the role of βarrs in signaling events elicited by the adhesion of Nm to endothelial cells, hCMEC/D3 cells, a human brain endothelial cell line, which stably maintains in culture phenotypic features of BBB in culture, was used as a model. Monolayers of hCMEC/D3 cells, inoculated with a capsulated piliated serogroup C Nm strain, displayed a characteristic reticulated immunofluorescent ezrin staining (used as a marker of cortical plaques) beneath 90% of bacterial colonies (data not shown). Cortical plaques were also previously reported to contain actin, cortactin, signaling molecules recruited in response to Nm adhesion, as well as components of both adhereris and tight junctions. In hCMEC/D3 cells expressing green fluorescent protein (GFP)-tagged βarr2 (βarr2-GFP), (βarr2-GFP colocalized with ezrin and bacteria, whereas in cells not in contact with Nm colonies or in non-infected monolayers βarr2-GFP displayed its expected diffuse cytosolic distribution, indicating that bacterial colonies are capable of locally translocating βarr2 . Similar results were obtained in cells expressing βarr1-GFP and for endogenous βarr2 (data not shown), hCMEC/D3 cells, which predominantly express βrra2 were then treated with small interfering RNAs (siRNAs) to establish whether βarr translocation to bacterial colonies occurs upstream or downstream of ezrin recruitment. Reduction of both βarr1 and βarr2 levels, via specific siRNAs, led to a decrease of ezrin-containing cortical plaques in an Nm adhesion assay (FIG. 1, left panel). The G protein receptor kinase (GRK) family specifically phosphorylates agonist-activated GPCRs, leading to their desensitization, GPCR phosphorylation by GRKs provides docking sites for βarrs. GRK2, a member of this family, was recruited under meningococcal colonies and its depletion inhibited both ezrin and βarr2 translocation under bacteria (FIG. 1, left and right panels). Control siRNA or siRNA directed against another signaling molecule, the heterotrimeric Gαₛ protein, did not affect cortical plaque formation (FIG. 1, left panel).

Altogether these data indicate that βarr translocation precedes ezrin recruitment, and are consistent with direct or indirect activation of a cell surface GPCR following tfp-mediated adhesion to endothelial cells.

### Example 3

### Nm Adhesion to Endothelial Cells Promotes βarr-Biased β2AR Signaling

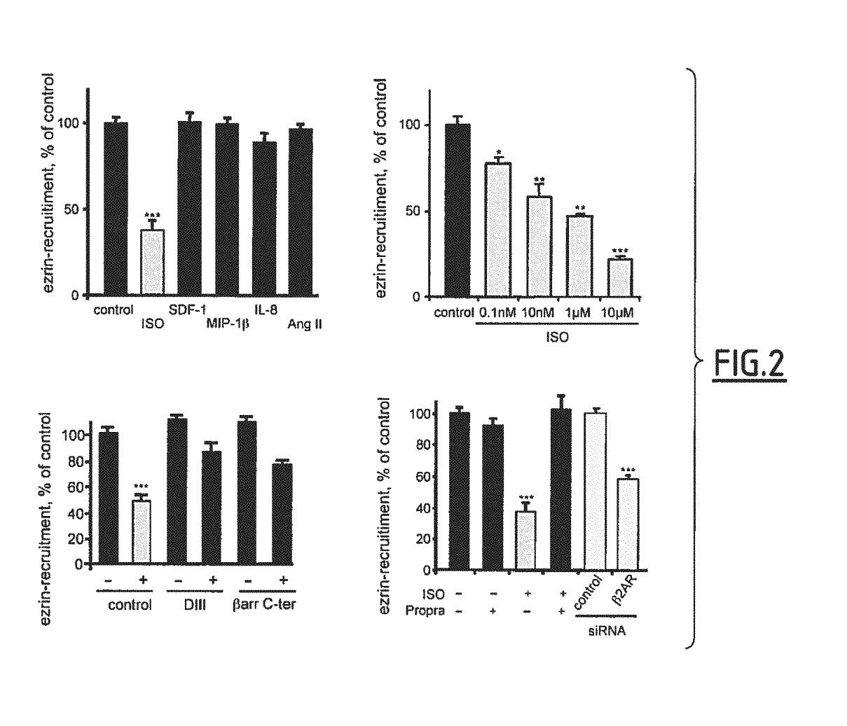

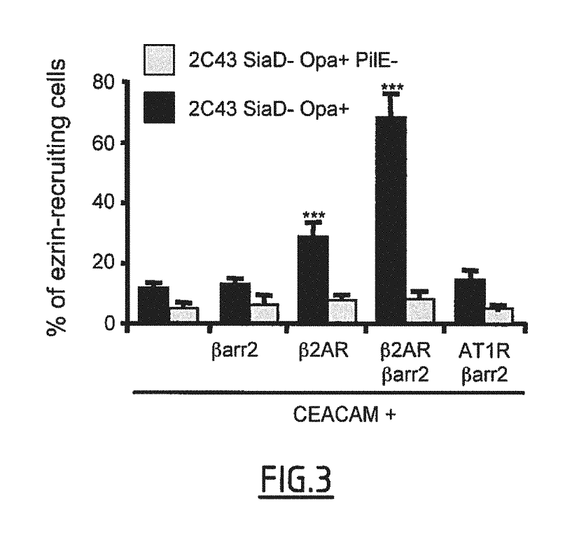

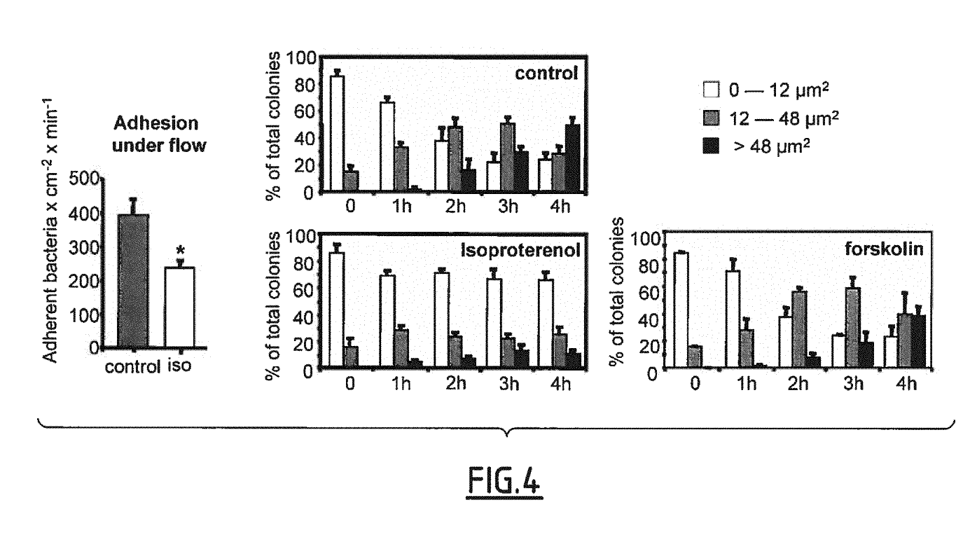

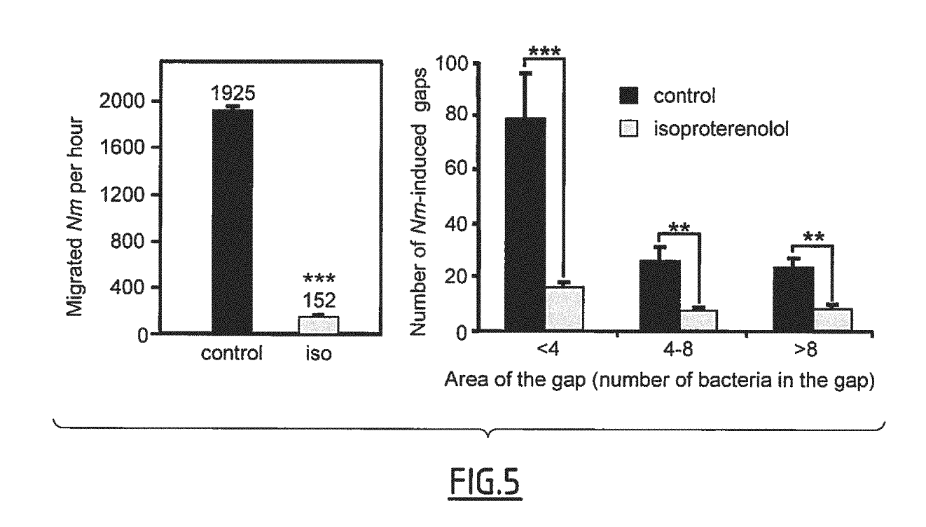

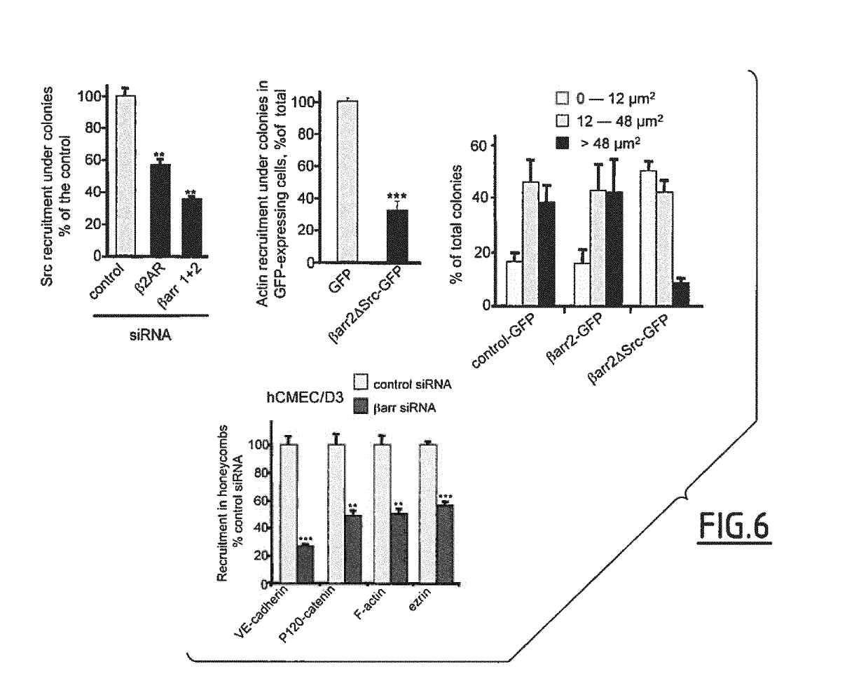

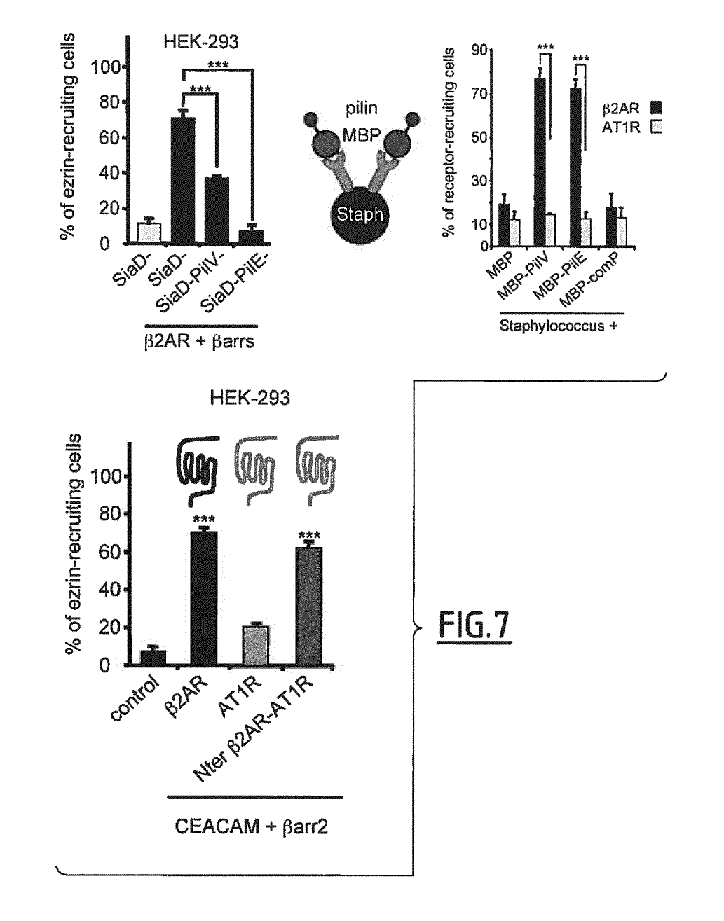

hCMEC/D3 cells naturally express several GPCRs, including β2ARs, angiotensin II AT1 receptors and various chemokine receptors. Sustained stimulation with agonists is known to induce GPCR desensitization and endocytosis, thus reducing their density at the cell surface. We reasoned that, if one of these receptors was activated by Nm to mediate βarr and ezrin translocation to Nm colonies, specific ligands of the receptor might interfere with this phenomenon. Various agonists were tested using the formation of ezrin honeycombs in hCMEC/D3 cells as readout. Cells were pre-treated before Nm inoculation with appropriate agonists for 2 h, a condition which often induces internalization and desensitization of cognate receptors. Among the compounds tested at saturating (10×K_{D}) concentrations, (FIG. 2, top left panel), only isoproterenol, a β2AR-specific agonist, showed an effect on ezrin recruitment. Moreover, isoproterenol elicited a dose-dependent reduction in the number of ezrin-containing cortical plaques (FIG. 2, top right panel). This effect was not due to a direct inhibition by isoproterenol of Nm growth in culture or of bacterial aggregation properties (data not shown). Also, isoproterenol pre-treatment did not reduce bacterial adhesion in static conditions (data not shown). In hCMEC/D3 cells expressing β2AR fused to YFP (β2AR-YFP), the formation of Nm colonies redistributed the receptor to ezrin-containing honeycombs (data not shown). in contrast, in hCMEC/D3 cells pretreated for 1 h with isoproterenol, β2AR-YFP was internalized in perinuclear endosomes and almost excluded from the small areas of residual ezrin below Nm colonies. To confirm that the inhibition on ezrin honeycomb formation by isoproterenol was due to β2AR internalization, a similar experiment was conducted in the presence of endocytosis inhibitors (FIG. 2, bottom left panel). The first, a specific inhibitor of the clathrin-dependent endocytic machinery, is a dominant negative mutant of Eps15 (Eps15-DIII. The second consists of the isolated carboxyterminal tail of βarr, which contains clathrin and AP2 binding sites and inhibits β2AR endocytosis. Supporting our hypothesis, hCMEC/D3 cells transfected with either mutant displayed a significantly reduced effect of isoproterenol on ezrin recruitment below the Nm colonies. The addition during the pre-incubation period of propranolol, a β2AR orthosteric antagonist, which inhibits receptor activation and endocytosis, blocked the effect of isoproterenol, as shown by the number of ezrin honeycombs, which remained comparable to that of untreated cells (FIG. 2, bottom right panel). Finally, siRNA directed against the β2AR in hCMEC/D3 cells, significantly decreased both the number of ezrin honeycombs (FIG. 2, bottom right panel) and βarr2 recruitment under bacterial colonies (FIG. 2, bottom left panel).

The data above indicate that the β2AR is involved in the early signaling events downstream of Nm adhesion.

GPCRs display various conformations, which are selectively stabilized, depending on the ligand that activates the receptor. Each conformation being competent for the activation of one or more effectors, different ligands can selectively activate or inhibit different subsets of effectors, leading to different signaling outputs. Activation of β2ARs with catecholamines or isoproterenol, for example, stimulates both the G protein Gαₛ-dependent activation of adenylyl cyclase and the Gαₛ-independent activation of βarrs. Activation of endogenous β2AR on hCMEC/D3 cells elicited a robust stimulation of cAMP production in response to isoproterenol, monitored by a cAMP response element (CRE) luciferase reporter, which was completely blocked by propranolol (data not shown). Incubation of hCMEC/D3 cells with Nm at a multiplicity of infection of 100 did not enhance the cAMP-dependent signal. Moreover, since the preincubation of hCMEC/D3 cells with saturating propranolol alone (FIG. 2, bottom right panel), or knockdown of Gαₛ by siRNA (data not shown) had no effect on the number of ezrin-containing cortical plaques elicited by Nm, the Nat-promoted stimulation of β2ARs appears to be biased toward the activation of the βarr pathway.

### Example 4

### Nm-Promoted Signaling Reconstituted in Infection-Incompetent Cells Via the Expression of Exogenous β2ARs and βarrs

To obtain additional evidence that the β2AR-βarr pathway is critical for the Nm-induced formation of cortical plaques in human cells, we investigated whether cells, which are not competent for the adhesion and signaling of Nm, can be switched to a permissive phenotype via the expression of exogenous proteins. HEK-293 cells were used as a model because they only express modest concentrations of endogenous βarrs and no β2ARs. Piliated capsulated Nm did not adhere onto HEK-293 cells (data not shown) and, consequently did not promote the formation of cortical plaques (data not shown). Transfection of the β2AR and/or βarr constructs in HEK -293 cells did not restore bacterial adhesion and/or signaling (data not shown), thus excluding that the β2AR on its own mediates tfp-dependent adhesion of capsulated Nm. However, since tfp-mediated adhesion and signaling are two independent events, the lack of initial bacterial adhesion could be responsible for the absence of signaling. Type IV pili are the only bacterial attributes capable of promoting adhesion of capsulated Nm. Yet, non-capsulated (SiaD−) strains can interact with host cells via Opa proteins, which recognize CEACAMs (carcinoembryonic antigen-related cell adhesion molecules), cellular receptors members of the Immunoglobulin superfamily. HEK-293 cells being devoid of CEACAMs, were thus transfected with a plasmid encoding CEACAM-1 and subsequently infected with a non-capsulated (SiaD−) Opa+ derivative of the piliated capsulated serogroup C strain used in the experiments above. Although the expression of CEACAM-1 allowed the development of piliated Nm colonies at the surface of HEK-293 cells, colonies did not induce significant recruitment of ezrin (data not shown). HEK-293 cells were then cotransfected with plasmids coding for CEACAM-1 and GFP (used as transfection marker) in the absence or presence of the β2AR and/or βarr2 plasmids, and the recruitment of ezrin assessed in GFP-positive cells. In the absence of exogenous β2AR and βarr2, only a low percentage of colonies recruited ezrin in microvilli-like structures with no visible honeycombs (FIG. 3). Transfection of the β2AR plasmid significantly raised the number of colonies (≈30%), which recruited ezrin in GFP-positive cells. The additional expression of exogenous βarr2 boosted the number of ezrin-recruiting colonies to nearly 70%, with typical honeycombs structures being visible in more than 50% of the cases (FIG. 3). In addition, the recruitment of ezrin was specifically promoted by the expression of the β2AR, as shown by the absence of ezrin recruitment above control values with expression of the AT1 angiotensin receptor, CCR5 or CXCR4 (FIG. 3). Interestingly, 3D reconstruction of confocal immunofluorescence images showed that ezrin accumulates both beneath and between meningococci within cellular protrusions similar to those observed in hCMEC/D3 cells infected with capsulated Nm (data not shown). Finally, cortical plaques in HEK-293 cells contained the exogenous β2AR and βarr2 as well as endogenous actin and p120 catenin (data not shown). In order to verify that tfp were responsible for the formation of the cortical plaque, similar experiments were performed using a non-piliated derivative (PilE−) of the non-capsulated Opa+ strain. This strain did not induce the formation of cortical plaques (data not shown).

These data confirm the involvement of the β2AR-βarr pathway in tfp-dependent signaling events, downstream of a β2ARsindependent adhesion of Nm to host cells.

### Example 5

### Nm Resistance to Shear Stress Requires Activation of a β2Ar-Dependent Pathway

Previous studies have shown that the bacteria-induced formation of the cortical plaque and of microvilli-like structures allow Nm to adhere to endothelial cells and resist to drag forces generated by bloodstream. To investigate the role of β2AR in the stable adhesion of Nm under experimental conditions recapitulating the pathophysiology of bacterial adhesion to brain endothelial cells, hCMEC/D3 cells were grown on IBIDI™ chambers and submitted to laminar flow under an inverted microscope. GFP-expressing bacteria were introduced in the chamber under controlled flow, and the number and size of adhering bacterial colonies counted over time. In control assays, bacterial colonies progressively increased in size, large colonies of 48 μm² or more (≥16 bacteria) representing about 50% of the total after 4 h, whereas small colonies (≤12 μm², about 4 bacteria) accounted for only 20% (FIG. 4, right panel). Preincubation of cells with isoproterenol for 1 h before the perfusion of bacteria, markedly reduced the average size of Nm colonies adhering to endothelial cells under flow: ≈70% of the colonies remained small throughout the experiment, large colonies barely representing 10% (FIG. 4, right panel). Interestingly, whereas the number of colonies forming in the absence of flow was similar in control conditions and after isoproterenol pre-treatment (data not shown), under constant flow the β2AR agonist caused a 40% decrease in the number of colonies forming (FIG. 4, left panel). These data suggest that, because of inhibited β-adrenergic signaling downstream of Nm adhesion, stable bacterial adhesion over time is impaired with larger colonies being progressively “wiped our” by the flow, a hypothesis supported by video data (data not shown).

Thus the activation of β2ARs appears a critical step for Nm to remain in strong interaction with brain endothelial cells for a sufficiently long period of time. Consistent with the involvement of a βarr-biased signaling pathway discussed above, pre-incubation with the adenylyl cyclase-stimulating agent forskolin, although inducing marked elevation of cAMP (data not shown) had no significant effect on the number and size of Nm colonies compared to control conditions. This result also eliminates the hypothesis of a cAMP-dependent modification of Nm adhesion to hCMEC/D3 cells that might have occurred during isoproterenol pre-incubation.

### Example 6

### Nm Crossing of Endothelial Cell Monolayers Involves β2Ars

Pili-mediated adhesion of capsulated Nm to brain endothelial cells is known to result in the depletion of junctional proteins at the cell-cell interface with a subsequent opening of the intercellular junctions. This depletion is caused by the recruitment of endothelial cell adhesion molecules, such as VE-cadherin, into cortical plaques. We examined the potential role of β2AR activation in these events using hCMEC/D3 cell monolayer grown on transwells as a model. First the number of bacteria passing through the monolayer was counted in control cells and in cells pre-incubated with isoproterenol. The β2AR agonist markedly reduced the total number of meningococci in the lower chamber of transwells (FIG. 5, left panel). Gaps induced by Nm on cell monolayers were directly visualized by confocal microscopy, and the area of the gaps was quantified using as surface unit the apparent diameter of a bacterium. Pre-incubation with isoproterenol noticeably reduced the number of the gaps of all size (FIG. 5, right panel).

Together, the above data demonstrate the involvement of the β2AR in Nm crossing of the brain endothelial barrier.

### Example 7

### Multiple Roles of βarrs in Cellular Signaling Events Promoted by Nm

The stabilization of bacteria at the cell surface of endothelial cells, which prevents their ejection by blood flow, requires cortactin-dependent formation of actin protrusions. Previous studies have shown that Src-dependent phosphorylation of cortactin is a key signaling intermediate in this context. Src and activated Src (phospho-Src) accumulated below Nm colonies in hCMEC/D3 cells (data not shown). Interestingly, it was reported previously that recruitment and activation of Src downstream of β-adrenoceptors can be mediated by βarrs, suggesting that Nm-promoted activation of Src may pass via βarrs. Supporting this hypothesis. siRNAs against either the β2AR or βarrs markedly reduced Src recruitment under colonies in hCMEC/D3 cells (FIG. 6, top left panel). Docking of Src to βarrs involves the interaction of Src homology 3 (SH3) domains with proline residues of the consensus sequence Pro-X-X-Pro (where X represents any amino acid) within the NH₂-terminus (position 1 to 185) of μarrs. It has been reported that point mutations of these praline residues, lead to a dominant negative form of βarr1 for Src activation, which conserved its capacity of interacting with the β-adrenoceptor and of mediating its endocytosis. Here we constructed the equivalent Src docking mutant in the context of βarr2 (myc-βarr2-ΔSrc or βarr2-ΔSrc-GFP) and examined its effect in Nm-promoted phosphorylation of Src and formation of actin-rich cellular protrusions. βarr2-ΔSrc-GFP markedly impaired recruitment of actin under Nm colonies in hCMEC/D3 cells, compared to control cells expressing GFP (FIG. 6, top middle panel). In addition, analysis of fluorescence images along the z-axis confirmed the marked reduction of actin-rich protrusions among bacteria (data not shown). Immunoblot experiments showed a reduction of phosphorylated Src after Nm infection in hCMEC/D3 cells expressing the dominant negative myc-βarr2-ΔSrc, compared to cells expressing myc-βarr2 (data not shown). The dominant negative effect of βarr2-ΔSrc was confirmed in hCMEC/D3 cells under flow. Expression of myc-βarr2-ΔSrc displayed the same effect on Nm colony size as the preincubation with isoproterenol (compare FIG. 6, top right panel and FIG. 4, right panel). βarrs might also participate in later events of Nm infection, namely the opening of gaps in the endothelial cell barrier.

This opening has been shown to involve the recruitment of junctional adhesion molecules, such as VE-cadherin and p120-catenin, into the cortical plaques, and their parallel depletion from endothelial cell junctions. We documented the interaction of βarrs with VE-cadherin and p120-catenin by co-immunoprecipitation experiments in both transfected HEK-293 cells and untransfected hCMEC/D3 (data not shown). In addition, the reduction of endogenous βarrs with specific siRNAs markedly and simultaneously affected the recruitment of VE-cadherin, p120-catenin. F-actin and ezrin into cortical plaques (FIG. 6, bottom panel), supporting the hypothesis that β2AR-mediated activation of βarrs provides a molecular connection between Nm colonies and the recruitment of junctional molecules.

### Example 8

### Molecular Determinants Involved in Nm β2Ar Interaction

Among the various GPCRs expressed in hCMEC/D3 cells, only the β2AR seems capable of mediating Nm signaling, implying some selective interaction between bacterial molecular determinants and a corresponding binding site on the β2AR. The fact that tfp are necessary for reconstituting signaling events in HEK-293 cells (FIG. 3) indicates that the putative β2AR “ligand” is present on these structures. Also in favor of this hypothesis are the consequences of the deletion of PilV, a supposed minor pilin component, which has been shown to impair the formation of the cortical plaque (Mikaty et al., 2009, PLoS Pathog 5, e1 000314), a finding confirmed in reconstituted HEK-293 cells (FIG. 7, top left panel).

To further characterize the supposed β2AR “ligand” at a molecular level, purified maltose binding protein (MBP)-fused pilins were bound to staphylococci coated with anti-MBP antibodies (FIG. 7, top right panel). In addition to PilE, two putative minor pilins, PilV and ComP, were tested. Whereas staphylococci coated with MBP alone or MBP fused to ComP were ineffective, staphylococci carrying either MBP-PilE or MBP-PilV promoted β2AR and βarr2 accumulation under staphylococcal aggregates (FIG. 7, top right panel). In control experiments, the AT1R was not recruited to bacteria. These data suggest that PilE and PilV can function as β2AR “ligands”.

Since Nm interaction with the β2AR does not involve the natural receptor binding-site (data in FIG. 2), the large β2AR extracellular N-terminus appears as a likely candidate region for receptor interaction with the pathogen. To test this hypothesis, we constructed a chimeric Nterβ2AR-AT1R receptor, in which the N-terminus of the β2AR replaced the equivalent portion of the incompetent AT1R. The chimeric receptor was indeed capable of mediating Nm-dependent signaling in the HEK-293 reconstituted model (FIG. 7, bottom panel), as did the β32AR. Altogether, these data suggest that pili components can directly interact with the N-teraninus of the β2AR.

In addition to conserved biological functions, such as surface mobility, adhesion or microcolony formation onto specific cell types, structural comparisons of the type IV pilins from different type of Gram-negative bacteria showed that all type IV pilins, despite some variably structured domains, share similar overall architecture (Craig et al., 2004, Nat Rev Microbiol 2, 363-378). We therefore examined whether Neisseria gonorrohae (Ng), a close relative of Nm which most often cause isolated infection of the genito-urinary tract but can, in rare cases, spread into the bloodstream and colonize meninges (Martin et al., 2008, Emerg Infect Dis 14, 672.674), might elicit similar signaling events to Nm in endothelial cells. The principal signaling events reported here for Nm, were also found in hCMEC/D3 cells infected by gonococci, demonstrating that the capacity of recruiting β-arrs via β2AR is not exclusive for Nm species (data not shown).

### Example 9

### Discussion

From our data, a more complete picture emerges of the signaling events elicited by Nm adhesion to human endothelial cells, leading to stable interaction with host cells and subsequent crossing of the endothelial monolayer. After binding onto target cells, Nm tfp interact with and activate the host cell β2AR, leading to the subsequent recruitment of GRK2 and βarrs, independently of the activation of the heterotrimeric Gαₛ protein, and of its downstream adenylyl cyclase-cAMP pathway. The selective activation of part of the signaling pathways downstream of a given GPCR, has been theorized and subsequently confirmed experimentally for synthetic ligands (Violin and Lefkowitz, 2007, Trends Pharmacol Sci 28, 416-422). However, this property, known as biased agonism, had not been described previously in a pathophysiologfcal context.

Once translocated to β2ARs, βarrs mediate at least 2 major events facilitating Nm infection. First, they dock and activate Src, leading to the formation of actin-rich cellular protrusions, which protect bacterial colonies against drag forces generated by the bloodstream. Second they provide sustained scaffolding for signaling events under Nm-activated β2ARs, which lead to the accumulation of βarr-interacting proteins, such as VE-cadherin and β120-catenin, into cortical plates under bacterial colonies. This accumulation eventually depletes intercellular junctions, which loosen and become permeable to bacteria. Although epithelial cells are the main target of Ng, gonococci possibly use the same pathway to infect brain endothelial cells in vivo in the rare cases of blood dissemination. It remains to be established whether other tfp-bearing bacteria capable of forming microcolonies at the surface of endothelial cells and crossing endothelial monolayers, can also hijack βarrs and/or β2ARs.

A few other examples of aberrant use of cellular GPCRs by pathogens to infect host cells exist, but the molecular mechanisms involved so far are quite different from those described here. For instance, Streptococcus pneumoniae is co-internalized with the platelet-activating factor receptor PAFr and delivered via transcytosis to the opposite basolateral membrane of endothelial cells. Also, the Human Immunodeficiency Virus, HIV, requires the association with CXCR4 or CCR5 chemokine receptors to infect cells. Binding of the viral gp120 to chemokine receptors, induces unmasking and insertion of a fusion peptide into the target cell membrane. Before interacting with chemokine receptors, HIV needs to bind to a second receptor, CD4.

Our experiments have shown that both sustained adhesion of Nm to endothelial cells under controlled flow and their crossing of endothelial cell monolayers by opening cellular junctions are markedly inhibited by pharmacological pre-activation and internalization of β2ARs. Thus, β2AR ligands, which induce β2AR endocytosis, might be beneficial in association with antibiotics to prevent or treat meningococcal infection.

In conclusion, signaling downstream of Nm adhesion to human brain endothelial cells, involves a biased activation of β2ARs and the subsequent βarr-dependent docking and activation of Src, which in turn activate the cortactin-dependent formation of cellular protrusions. Subsequently, βarrs scaffold cytoskeletal and junctional proteins under stabilized bacterial colonies, which progressively weakens the endothelial barrier until anatomical breakage occurs. This scenario represents a novel mechanism by which a pathogen hijacks cellular signaling machineries to infect target tissues. Fortuitously, the signaling pathway involved in this scenario can be targeted by clinically approved drugs, which could now be tested as adjuvant therapies, not only in the context of Nm meningitis but also possibly for meningococcal sepsis, the most devastating form of Nm infection, which is characterized by diffuse endothelial leakage and subsequent irreversible shock.

1. A method for preventing the spreading of meningococci into the meningeal space in a subject comprising the step of administering to a subject in need thereof an effective amount of a meningococcal PilE or PilV protein, or an immunogenic fragment of at least 10 consecutive amino acids thereof.

2. The method according to claim 1, wherein:
said PilE protein comprises or consists of the sequence SEQ ID NO: 1 or a sequence at least 80% identical to SEQ ID NO:1,
said PilV protein comprises or consists of the sequence SEQ ID NO: 2 or a sequence at least 80% identical to SEQ ID NO:2,
said immunogenic fragment of at least 10 consecutive amino acids of PilE or PilV comprises or consists of the recognition domain to the mammalian β2 adrenoreceptor.

3. The method according to claim 1, wherein said meningococcal PilE or PilV protein, or immunogenic fragment of at least 10 consecutive amino acids thereof induces the production of protective antibodies inhibiting the interaction between PilE and/or PilV and mammalian β2 adrenoreceptor.

4. A combination of at least two of:
a meningococcal PilE protein, or an immunogenic fragment of at least 10 consecutive amino acids thereof,
a meningococcal PilV protein, or an immunogenic fragment of at least 10 consecutive amino acids thereof; and
a meningococcal vaccine antigen,
wherein said meningococcal vaccine antigen is not PilE or PilV or an immunogenic fragment thereof.

5. A combination according to claim 4, wherein:
said PilE protein comprises or consists of the sequence SEQ ID NO: 1 or a sequence at least 80% identical to SEQ ID NO:1,
said PilV protein comprises or consists of the sequence SEQ ID NO: 2 or a sequence at least 80% identical to SEQ ID NO:2,
said immunogenic fragment of at least 10 consecutive amino acids of PilE or PilV comprises or consists of the recognition domain to the mammalian β2 adrenoreceptor.

6. A method for preventing a meningococcal infection in a subject comprising the step of administering to a subject in need thereof an effective amount of a combination of at least two of:
a) a meningococcal PilE protein, or an immunogenic fragment of at least 10 consecutive amino acids thereof;
b) a meningococcal PilV protein, or an immunogenic fragment of at least 10 consecutive amino acids thereof; and
c) a meningococcal vaccine antigen;
wherein said meningococcal vaccine antigen is not a PilE, PilV, or an immunogenic fragment thereof.

7. The method according to claim 6, wherein:
said PilE protein comprises or consists of the sequence SEQ ID NO: 1 or a sequence at least 80% identical to SEQ ID NO:1,
said PilV protein comprises or consists of the sequence SEQ ID NO: 2 or a sequence at least 80% identical to SEQ ID NO:2,
said immunogenic fragment of at least 10 consecutive amino acids of PilE or PilV comprises or consists of the recognition domain to the mammalian β2 adrenoreceptor.

8. The method according to claim 6, wherein said combination induces the production of protective antibodies inhibiting the interaction between PilE and/or PilV and mammalian β2 adrenoreceptor.

9. A fusion protein comprising at least two of:
a) a meningococcal PilE protein, or an immunogenic fragment of at least 10 consecutive amino acids thereof;
b) a meningococcal PilV protein, or an immunogenic fragment of at least 10 consecutive amino acids thereof; and
c) a meningococcal vaccine antigen,
wherein said meningococcal vaccine antigen is not PilE, PilV, or an immunogenic fragment thereof.

10. A fusion protein according to claim 9, wherein:
said PilE protein comprises or consists of the sequence SEQ ID NO: 1 or a sequence at least 80% identical to SEQ ID NO:1,
said PilV protein comprises or consists of the sequence SEQ ID NO: 2 or a sequence at least 80% identical to SEQ ID NO:2,
said immunogenic fragment of at least 10 consecutive amino acids of PilE or PilV comprises or consists of the recognition domain to the mammalian β2 adrenoreceptor.

11. A fusion protein according to claim 9, wherein said fusion protein induces the production of protective antibodies inhibiting the interaction between PilE and/or PilV and mammalian β2 adrenoreceptor.

12. A method for preventing a meningococcal infection in a subject comprising the step of administering to a subject in need thereof an effective amount of a fusion protein comprising at least two of:
a) a meningococcal PilE protein, or an immunogenic fragment of at least 10 consecutive amino acids thereof;
b) a meningococcal PilV protein, or an immunogenic fragment of at least 10 consecutive amino acids thereof; and
c) a meningococcal vaccine antigen,
wherein said meningococcal vaccine antigen is not PilE, PilV, or an immunogenic fragment thereof.

13. The method according to claim 12, wherein:
said PilE protein comprises or consists of the sequence SEQ ID NO: 1 or a sequence at least 80% identical to SEQ ID NO:1,
said PilV protein comprises or consists of the sequence SEQ ID NO: 2 or a sequence at least 80% identical to SEQ ID NO:2,
said immunogenic fragment of at least 10 consecutive amino acids of PilE or PilVcomprises or consists of the recognition domain to the mammalian β2 adrenoreceptor.

14. The method according to claim 12, wherein said fusion protein induces the production of protective antibodies inhibiting the interaction between PilE and/or PilV and mammalian β2 adrenoreceptor.

15. A method for preventing a meningococcal infection in a subject comprising the step of administering to a subject in need thereof an effective amount of a vaccine composition or an immunogenic composition, wherein said vaccine or immunogenic composition comprises or consists of:
a meningococcal PilE or PilV protein, or an immunogenic fragment of at least 10 consecutive amino acids thereof,
a combination of at least two of:
a meningococcal PilE protein, or an immunogenic fragment of at least 10 consecutive amino acids thereof,
a meningococcal PilV protein, or an immunogenic fragment of at least 10 consecutive amino acids thereof; and
a meningococcal vaccine antigen, wherein said meningococcal vaccine antigen is not PilE or PilV or an immunogenic fragment thereof, or
a fusion protein according to claim 9.


SEQUENCE LISTING (30 sequences)

SEQ ID NO: 1 - protein, 168 aa, Neisseria meningitidis
MNTLQKGFTLIELMIVIAIVGILAAVALPAYQDYTARAQVSEAILLAEGQKSAVTEYYLNHGEWPGDNSSAGVATSADIKGKYVQSVTVANGVITAQMASSNVNNEIKSKKLSLWAKRQNGSVKWFCGQPVTRTTATATDVAAANGKTDDKINTKHLPSTCRDDSSAS

SEQ ID NO: 2 - protein, 129 aa, Neisseria meningitidis
MKNVQKGFTLLELMIAVAILGILTLITYPSYKTYIRRVRLSEVRTTLLHNAQTMERYYRQKGTFKTYDKNKLKQNKYFNVTLSKVSPDHFTLQADPNPTTNDGETCVVTLNDGGTIAASGTNQSCPGFD

SEQ ID NO: 3 - protein, 413 aa, Homo sapiens [sequence omitted]

SEQ ID NO: 4 - RNA, 21 nt, Artificial
  Synthetic siRNA
aaagccuucugcgcggagaau

SEQ ID NO: 5 - RNA, 21 nt, Artificial
  Synthetic siRNA
ggcuuguccuuccgcaaagac

SEQ ID NO: 6 - RNA, 23 nt, Artificial
  Synthetic siRNA
aagaaguacgagaagcuggaguu

SEQ ID NO: 7 - DNA, 21 bp, Artificial
  Synthetic offtarget siRNA
gataaggtgctgcgtggaccc

SEQ ID NO: 8 - DNA, 25 bp, Artificial
  Synthetic oligonucleoctide primer
gaattccggcgcgtccgcctgtcgg

SEQ ID NO: 9 - DNA, 26 bp, Artificial
  Synthetic oligonucleoctide primer
ctgcagttagtcgaagccggggcagg

SEQ ID NO: 10 - DNA, 26 bp, Artificial
  Synthetic oligonucleoctide primer
gaattcgagaaagcaaagataaatgc

SEQ ID NO: 11 - DNA, 28 bp, Artificial
  Synthetic oligonucleoctide primer
ctgcagctacttaaataacttgcagtcc

SEQ ID NO: 12 - DNA, 20 bp, Artificial
  Synthetic oligonucleoctide primer
attgagaccctgtgcgtgat

SEQ ID NO: 13 - DNA, 20 bp, Artificial
  Synthetic oligonucleoctide primer
ctgaaagaccctggagtaga

SEQ ID NO: 14 - DNA, 20 bp, Artificial
  Synthetic oligonucleoctide primer
ttcctacccccaatgtgtcc

SEQ ID NO: 15 - DNA, 20 bp, Artificial
  Synthetic oligonucleoctide primer
tcttgctcagtgtccttgct

SEQ ID NO: 16 - protein, 52 aa, Neisseria meningitidis
FTLIELMIVIAIVGILAAVALPAYQDYTARAQVSEAILLAEGQKSAVTEYYL

SEQ ID NO: 17 - protein, 53 aa, Neisseria meningitidis
FTLLELMIAVAILGILTLITYPSYKTYIRRVRLSEVRTTLLHNAQTMERYYRQ

SEQ ID NO: 18 - protein, 117 aa, Neisseria meningitidis
SAVTEYYLNHGEWPGDNSSAGVATSADIKGKYVQSVTVANGVITAQMASSNVNNEIKSKKLSLWAKRQNGSVKWFCGQPVTRTTATATDVAAANGKTDDKINTKHLPSTCRDDSSAS

SEQ ID NO: 19 - protein, 76 aa, Neisseria meningitidis
TMERYYRQKGTFKTYDKNKLKQNKYFNVTLSKVSPDHFTLQADPNPTTNDGETCVVTLDGGTIAASGTNQSCPGFD

SEQ ID NO: 20 - protein, 35 aa, Neisseria meningitidis
CGQPVTRTTATATDVAAANGKTDDKINTKHLPSTC

SEQ ID NO: 21 - protein, 27 aa, Neisseria meningitidis
GETCVVTLNDGGTIAASGTNQSCPGFD

SEQ ID NO: 22 - protein, 280 aa, Neisseria meningitidis
MTRSKPVNRTAFCCLSLTAALILTACSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 23 - protein, 279 aa, Neisseria meningitidis
MTRSKPVNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 24 - protein, 395 aa, Neisseria meningitidis
MRKKLTALVLSALPLAAVADVSLYGEIKAGVEGRNYQLQLTEAQAANGGASGQVKVTKVTKAKSRIRTKISDFGSFIGFKGSEDLGEGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPAQNSKSAYKPAYWTTVNTGSATTTTFVPAVVGKPGSDVYYAGLNYKNGGFAGNYAFKYARHANVGRDAFELFLLGSGSDQAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 25 - protein, 497 aa, Neisseria meningitidis
MFKRSVIAMACIVALSACGGGGGGSPDVKSADTLSKPAAPVVTEDVGEEVLPKEKKDEEAVSGAPQADTQDATAGKGGQDMAAVSAENTGNGGAATTDNPENKDEGPQNDMPQNAADTDSSTPNHTPAPNMPTRDMGNQAPDAGESAQPANQPDMANAADGMQGDDPSAGENAGNTADQAANQAENNQVGGSQNPASSTNPNATNGGSDFGRINVANGIKLDSGSENVTLTHCKDKVCDRDFLDEEAPPKSEFEKLSDEEKINKYKKDEQRENFVGLVADRVEKNGTNKYVIIYKDKSASSSSARFRRSARSRRSLPAEMPLIPVNQADTLIVDGEAVSLTGHSGNIFAPEGNYRYLTYGAEKLSGGSYALSVQGEPAKGEMLAGTAVYNGEVLHFHMENGRPSPSGGRFAAKVDFGSKSVDGIIDSGDDLHMGTQKFKAVIDGNGFKGTWTENGGGDVSGRFYGPAGEEVAGKYSYRPTDAEKGGFGVFAGKKEQD

SEQ ID NO: 26 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 27 - protein, 396 aa, Artificial
  Synthetic fusion protein PilE-fHBP
SAVTEYYLNHGEWPGDNSSAGVATSADIKGKYVQSVTVANGVITAQMASSNVNNEIKSKKLSLWAKRQNGSVKWFCGQPVTRTTATATDVAAANGKTDDKINTKHLPSTCRDDSSASMTRSKPVNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 28 - protein, 314 aa, Artificial
  Synthetic fusion protein PilE-fHBP
CGQPVTRTTATATDVAAANGKTDDKINTKHLPSTCMTRSKPVNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 29 - protein, 355 aa, Artificial
  Synthetic fusion protein PilV-fHBP
TMERYYRQKGTFKTYDKNKLKQNKYFNVTLSKVSPDHFTLQADPNPTTNDGETCVVTLDGGTIAASGTNQSCPGFDMTRSKPVNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 30 - protein, 306 aa, Artificial
  Synthetic fusion protein PilV-fHBP
GETCVVTLNDGGTIAASGTNQSCPGFDMTRSKPVNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ